(19) United States
(12) Patent Application Publication
(10) Pub. No.: US 2020/0271643 A1
(43) Pub. Date: Aug. 27, 2020

(54) METHODS AND SYSTEMS FOR THE DETECTION OF ANALYTE MOLECULES

(71) Applicant: Quanterix Corporation, Billerica, MA (US)

(72) Inventors: David Wilson, Boxborough, MA (US); David M. Rissin, Somerville, MA (US); Lei Chang, Winchester, MA (US); Andrew Rivnak, Somerville, MA (US); David C. Duffy, Arlington, MA (US)

(73) Assignee: Quanterix Corporation, Billerica, MA (US)

(21) Appl. No.: 16/648,788

(22) PCT Filed: Sep. 20, 2018
(86) PCT No.: PCT/US2018/052040

§ 371 Date: Mar. 19, 2020

Related U.S. Application Data
(60) Provisional application No. 62/561,169, filed on Sep. 20, 2017.

(51) Int. Cl. G01N 33/542; G01N 33/53; G01N 33/573

(52) U.S. Cl. CPC G01N 33/542; G01N 33/573; G01N 33/5306

(57) ABSTRACT

Methods and systems for the detection of analyte molecules are generally described. In some embodiments, a method may comprise using a stabilizing agent prior to, during, and/or after one or more steps of a detection assay. The stabilizing agent may serve to maintain and/or increase the detectable signal indicative of the analyte during one or more assay steps and/or until the signal is measured. In such cases, the stabilizing agent may reduce and/or prevent one or more phenomena associated with signal decay, such as, e.g., dissociation between, aggregation of, and/or denaturation of analyte molecules and/or detection molecules. In some embodiments, the stabilizing agent can be used to improve the sensitivity of new and existing assays with little or no adverse effect on specificity. The methods and systems, described herein, may be used for a plethora of applications, including the detection or quantification of low levels of analyte molecules.

### FIELD OF THE INVENTION

Methods and systems for the detection of analyte molecules are generally described.

### BACKGROUND OF THE INVENTION

Methods and systems that are able to quickly and accurately detect and, in certain cases, quantify a target analyte molecule in a sample are the cornerstones of modern analytical measurements. Such systems and/or methods are employed in many areas such as academic and industrial research, environmental assessment, food safety, medical diagnosis, and detection of chemical, biological, and/or radiological warfare agents. Advantageous features of such techniques may include specificity, speed, and sensitivity.

Most current techniques for quantifying low levels of analyte molecules in a sample use amplification procedures to increase the number of reporter molecules in order to be able to provide a measurable signal. For example, these known processes include enzyme-linked immunosorbent assays (ELISA) for amplifying the signal in antibody-based assays, as well as the polymerase chain reaction (PCR) for amplifying target DNA strands in DNA-based assays. One feature of typical known methods and/or systems for detecting or quantifying low concentrations of a particular analyte molecule in solution is that they are based on ensemble responses in which many analyte molecules give rise to a measured signal. Most detection schemes require that a large number of molecules are present in the ensemble for the aggregate signal to be above the detection threshold. This requirement limits the sensitivity of most detection techniques and the dynamic range (i.e., the range of concentrations that can be detected). Many of the known methods and techniques are further plagued with problems of non-specific binding, which is the binding of analyte molecules or particles to be detected or reporter species non-specifically to sites other than those expected. This leads to an increase in the background signal, and therefore limits the lowest concentration that may be accurately or reproducibly detected.

Accordingly, improved methods for detecting and, optionally, quantifying analyte molecules are needed, especially in samples where such molecules are present at very low concentration.

### SUMMARY OF THE INVENTION

Methods and systems for the detection of analyte molecules are generally described. The subject matter of the present invention involves, in some cases, interrelated products, alternative solutions to a particular problem, and/or a plurality of different uses of one or more systems and/or articles.

In one set of embodiments, methods are provided. In one embodiment a method for perform an assay comprises combining a stabilizing agent and an assay composition to form a first liquid mixture comprising a liquid, wherein the assay composition comprises detection molecules associated with analyte molecules. The method further comprises separating at least a portion of a liquid from at least a portion of the first liquid mixture to form a pellet and a separated liquid, wherein the pellet comprises at least a portion of the assay composition and at least a portion of the stabilizing agent. The method further comprises removing greater than or equal to about 95% of the separated liquid from the pellet, wherein the stabilizing agent comprises a molecule having greater than four hydroxyl groups.

In another embodiment, a method for preventing or reducing signal decay in an assay comprises combining a stabilizing agent with an assay composition to form a first liquid mixture, wherein the assay composition comprises detection molecules associated with analyte molecules; removing greater than or equal to about 95% of a liquid from the first liquid mixture to form a concentrated composition comprising the assay composition and the stabilizing agent; and storing the concentrated composition for at least 1 minute. The method further comprises reconstituting the concentrated composition to form a second liquid mixture and measuring a signal used to determine a concentration of the analyte molecules in the second liquid mixture, wherein a magnitude of the signal measured in the measuring step is greater than or equal to about 10% of the magnitude of a signal used to determine a concentration of the analyte molecules of the analyte molecules in an otherwise identically prepared second liquid mixture except not subjected to the storage step.

Other advantages and novel features of the present invention will become apparent from the following detailed description of various non-limiting embodiments of the invention when considered in conjunction with the accompanying figures. In cases where the present specification and a document incorporated by reference include conflicting and/or inconsistent disclosure, the present specification shall control.

### BRIEF DESCRIPTION OF THE DRAWINGS

Other aspects, embodiments, and features of the invention will become apparent from the following detailed description when considered in conjunction with the accompanying drawings. The accompanying figures are schematic and are not intended to be drawn to scale. For purposes of clarity, not every component is labeled in every figure, nor is every component of each embodiment of the invention shown where illustration is not necessary to allow those of ordinary skill in the art to understand the invention. All patent applications and patents mentioned in the text are incorporated by reference in their entirety. In case of conflict between the description contained in the present specification and a document incorporated by reference, the present specification, including definitions, will control.

FIG. 1A is a schematic flow diagram depicting a detection method without a stabilizing agent, according to certain embodiments;

FIG. 1B is a schematic flow diagram depicting a detection method with a stabilizing agent, according to certain embodiments;

FIG. 2A is a schematic flow diagram depicting a detection method without a stabilizing agent, according to certain embodiments;

FIG. 2B is a schematic flow diagram depicting a detection method with a stabilizing agent, according to certain embodiments;

FIG. 2C is a schematic flow diagram depicting a detection method with a stabilizing agent, according to certain embodiments;

FIG. 3A is a schematic flow diagram depicting a detection method without a stabilizing agent, according to certain embodiments;

FIG. 3B is a schematic flow diagram depicting a detection method with a stabilizing agent, according to certain embodiments;

FIG. 3C is a schematic flow diagram depicting a detection method with a stabilizing agent, according to certain embodiments; and

FIG. 4 is a graph of average intensity versus time for a detection method, according to certain embodiments;

FIG. 5 is a graph of average enzyme per bead (AEB) versus time for a detection method, according to certain embodiments;

FIG. 6 is a graph of average intensity versus time for a detection method, according to certain embodiments;

FIG. 7 is a histogram of average enzyme per bead (AEB) for various assay mixtures, according to certain embodiments;

FIG. 8 is a histogram of average enzyme per bead (AEB) for various assay mixtures, according to certain embodiments;

FIG. 9 is a graph of average enzyme per bead (AEB) versus percent sucrose, according to certain embodiments;

FIG. 10 is a graph of percent bead fill versus percent sucrose, according to certain embodiments;

FIG. 11 is a graph of normalized average enzyme per bead (AEB) versus time, according to certain embodiments;

FIG. 12A is a schematic flow diagram depicting a detection method without a stabilizing agent, according to certain embodiments; and

FIG. 12B is a schematic flow diagram depicting a detection method with a stabilizing agent, according to certain embodiments.

### DETAILED DESCRIPTION

Methods and systems for the detection of analyte molecules are generally described. In some embodiments, a method may comprise using a stabilizing agent (e.g., polyol, carbohydrate, disaccharide) prior to, during, and/or after one or more steps of an assay. The stabilizing agent may serve to maintain and/or increase a detectable signal indicative of (e.g., presence of, absence of, quantity of) an analyte molecule during one or more assay steps and/or until the signal is measured. In such cases, the stabilizing agent may reduce and/or prevent one or more phenomena associated with signal decay, such as, e.g., dissociation between, aggregation of, and/or denaturation of analyte molecules and/or detection molecules. For example, the stabilizing agent may prevent dissociation between analyte molecules and detection molecules during storage and/or desolvation. In some embodiments, the stabilizing agent can be used to improve the sensitivity (e.g., detection limit) of new and existing assays with little or no adverse effect on specificity. The methods and systems, described herein, may be used for a plethora of applications, including the detection or quantification of low levels of analyte molecules.

In some embodiments, the assay is a detection assay. Detection assays often rely on the measurement of a signal indicative of an analyte molecule to determine the presence, absence, and/or quantity of the analyte. However, many analyte molecules do not produce such a signal and/or do not produce a detectable signal at certain concentrations. Accordingly, many detection assays utilize detection molecules that specifically (e.g., exclusively) associate with the analyte molecules and can be used to produce and/or amplify a signal indicative of the analyte molecules. Many such detection assays require steps, such as manipulation of the analyte molecule after association with the detection molecules (e.g., drying, suspension, washing, transfer), which results in a delay before measurements that can result in a change in the signal. This change in the signal may reduce the sensitivity and/or dynamic range of the assay. The delay between exposure of the analyte molecule to detection molecules and signal measurement (e.g., the time in which a signal of the detection molecules is determined) often results in a signal reduction. For example, in embodiments in which the detection molecule is an antibody and the analyte molecule is an antigen, a time delay between exposure and measurement may allow dissociation to occur between the detection molecule and analyte molecule. The amount of dissociation between the detection molecule and analyte molecule may increase with time, such that the signal measured after a delay is significantly lower than the signal measured immediately after exposure to the detection molecules. This reduction in the signal can reduce the sensitivity and/or accuracy of the assay. Therefore, it is desirable to reduce and/or eliminate any change in the signal.

The time delay between exposure and measurement may also adversely affect calibration curves and comparative analysis of samples. In some embodiments, the time delay between exposure and determining the signal often has greater impact when the signal measured is in or being compared to a calibration curve. For instance, a calibration curve produced from samples having different time delays between exposure and measurement may, in certain instances, be inaccurate. Moreover, even if the calibration curve was produced from samples having the same time delay, use of the calibration curve for samples measured with a different time delay may result in inaccuracies. In some assays, analysis requires comparison of multiple samples. In such assays, multiple samples are prepared at a single time point (e.g., a plurality of samples are exposed to detection molecules at the same time), but only a single sample can be analyzed at a time. Thus, the signal of the first sample would be determined at a much earlier time point following exposure to the detection molecule compared to another sample. Variation in time delay between replicates of the same sample can result in imprecision and high coefficient of variation of the measurement, i.e., there will be variability in the signal from test-to-test. This variation can lead to imprecision within a run, run-to-run, day-to-day, instrument-to-instrument, site-to-site, and user-to-user, for examples.

Existing methods have tried to address this problem by ensuring that every sample experiences the same time delay (e.g., the same timing of incubation, washing, and detection during an experiment). In this way, the time between exposure and measurement is fixed. Thus, any change in the signal due to the delay should be similar for all samples and is not expected to significantly affect the precision of the assay. However, in practice, this approach means that each sample has to be processed individually with the same delays, and/or all processed simultaneously. Thus, while the difference in time between exposure and determination could be minimized by keeping the time delay constant for each sample (e.g., sample 1 is exposed to detection molecules at time point X1 and the signal for sample 1 is determined Y minutes after time point X1, and sample 2 is exposed to detection molecules at time point X2 and the signal for sample 2 is determined Y minutes after time point X2, wherein time point X1 and X2 are different), control of the exposure and determination for any significant number of samples would be time consuming and is not possible with many automated assay systems and methods. Existing methods have also tried to address the problem of differences in time delay by applying a mathematical correction to the data to correct for differences in the time between exposure and measurement. While this approach is theoretically feasible, it is dependent on the change in the signal over time being stable and predictable, which is often not true in practice. Experimental variation in the change in the signal over time occur limiting the utility of the mathematical approach. Accordingly, improved methods for reducing and/or minimizing the change in signal due to time delays are needed.

The inventors have developed in the context of the present invention techniques and compositions that employ certain stabilizing agents to reduce and/or prevent signal decay when used prior to, during, and/or after certain assay steps. In some embodiments, polyols, such as sucrose and trehalose, may be used to reduce and/or prevent one or more phenomena associated with signal decay, such as dissociation, reduction of a measurable property of the detection molecules (e.g., enzymatic activity), aggregation, and/or denaturation associated with certain assay steps (e.g., desolvation, suspension, storage, prolonged time until measurement). Without being bound by theory, it is believed that the stabilizing agents, described herein, may maintain the structure that proteins and protein complexes form in aqueous solutions without water being necessarily present. In some embodiments, this stabilization of structure is achieved by stabilizing agents that maintain the hydrogen bond network between the surface of proteins and water so that they do not denature. For example, the stabilizing agent may be a non-ionic kosmotrope (e.g., trehalose and sucrose). Non-ionic kosmotropes have no net charge and strongly hydrogen bonding to water. The non-ionic kosmotropes may be used as a stabilizing agent to stabilize the structure of proteins and other biological molecules in solution. Without being bound by theory, it is believed that kosmotropes reduce the volume of water available to hydrate the larger surface exposed by denatured proteins, which tends to prevent the denaturation process. In some embodiments, the stabilizing agents, described herein, may have two or more (e.g., three or more, four or more, five or more, six or more) hydrogen bond donors and/or hydrogen bond acceptors. For example, the stabilizing agent may be a polyol or a sugar. In certain embodiments, the stabilizing agent may be a zwitterion kosmotrope. The zwitterion kosmotrope may have a similar stabilizing effects as the non-ionic kosmotrope. The methods and systems, described herein, may be used to improve the sensitivity and/or dynamic range of a wide variety of new and existing assays.

In some embodiments, a method for performing an assay is provided comprising combining a stabilizing agent and an assay composition (e.g., comprising analyte molecules associated with detection molecules) to form a first liquid mixture comprising a liquid. The assay composition may additionally comprise capture objects (e.g., beads) associated with the analyte molecules. At least a portion of a liquid from at least a portion of the first liquid mixture may be separated to form a pellet and a separated liquid, wherein the pellet comprises at least a portion of the assay composition and at least a portion of the stabilizing agent. Greater than or equal to about 95% of the separated liquid may then be removed from the pellet, wherein the stabilizing agent comprises a molecule having greater than four hydroxyl groups.

In other embodiments, a method for preventing or reducing signal decay in an assay is provided. In some embodiments, a stabilizing agent is combined with an assay composition to form a first liquid mixture, wherein the assay composition comprises detection molecules associated with analyte molecules. Greater than or equal to about 95% of a liquid from the first liquid mixture may be removed to form a concentrated composition (e.g., a pellet) comprising the assay composition and the stabilizing agent. The concentrated composition (e.g. pellet) may be stored for any desired period of time (e.g., for at least 5 seconds, for at least 30 seconds, for at least 1 minute, for at least 10 minutes, for at least 30 minutes, for at least 60 minutes, etc.). The concentrated composition (e.g., pellet) may be reconstituted to form a second liquid mixture. For example, a liquid may be added to the concentrated composition. The amount of liquid added may be the same amount of liquid that was removed during the removing step. A signal used to determine a concentration of the analyte molecules in the second liquid mixture may then be determined. In some cases, a magnitude of the signal measured in the measuring step is greater than or equal to about 10% (e.g., greater than or equal to about 20%, greater than or equal to about 30%, greater than or equal to about 40%, greater than or equal to about 50%, greater than or equal to about 60%, greater than or equal to about 70%, greater than or equal to about 80%, greater than or equal to about 85%, greater than or equal to about 90%, greater than or equal to about 95%) of a magnitude of a signal used to determine a concentration of the analyte molecules of the analyte molecules in an otherwise identically prepared second liquid mixture except not subjected to the storage step. In certain embodiments, the magnitude of the signal measured in the measuring step is greater than or equal to about 80% (greater than or equal to about 85%, greater than or equal to about 90%, greater than or equal to about 95%) of a magnitude of a signal used to determine a concentration of the analyte molecules of the analyte molecules in an otherwise identically prepared second liquid mixture except not subjected to the storage step.

In some embodiments wherein capture objects are employed, the pellet or concentrated composition comprises greater than about 50%, or greater than about 60%, or greater than about 70%, or greater than about 80%, or greater than about 90%, or greater than about 95%, or more, of the capture objects initially provided. In some embodiments, the pellet or concentrated composition comprises at least 1000 capture objects, or at least 2000 capture objects, or at least 3000 capture objects, or at least 5000 capture objects, or at least 10,000 capture objects, or at least 20,000 capture objects, or at least 30,000 capture objects, or at least 50,000 capture objects, or at least 100,000 capture objects.

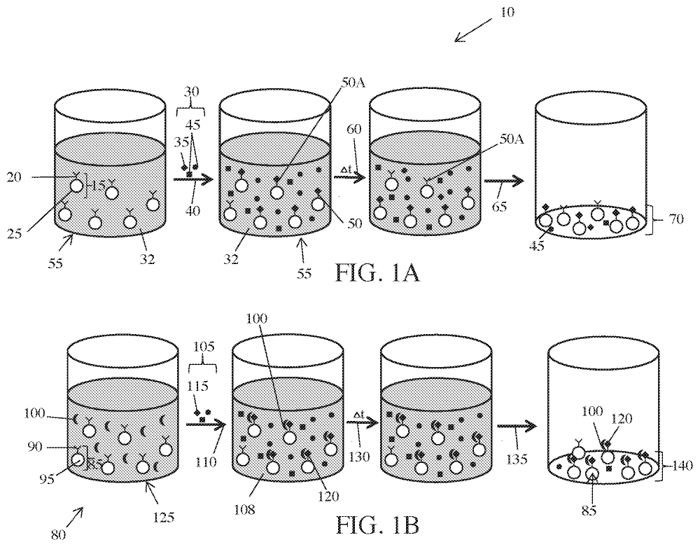

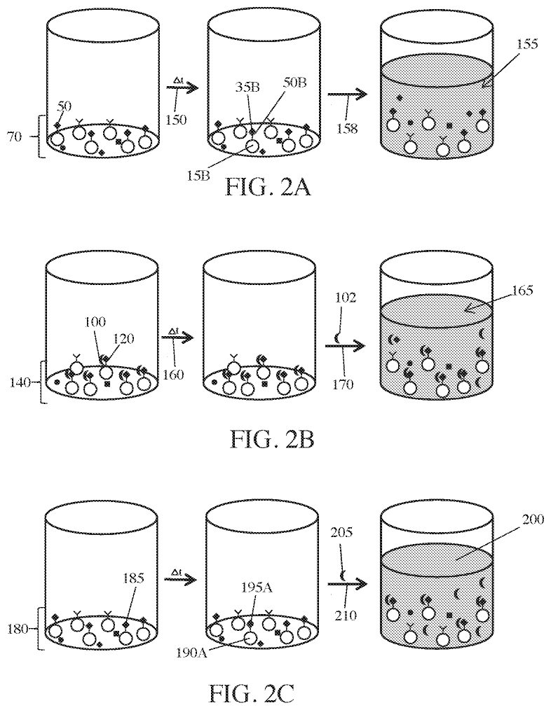

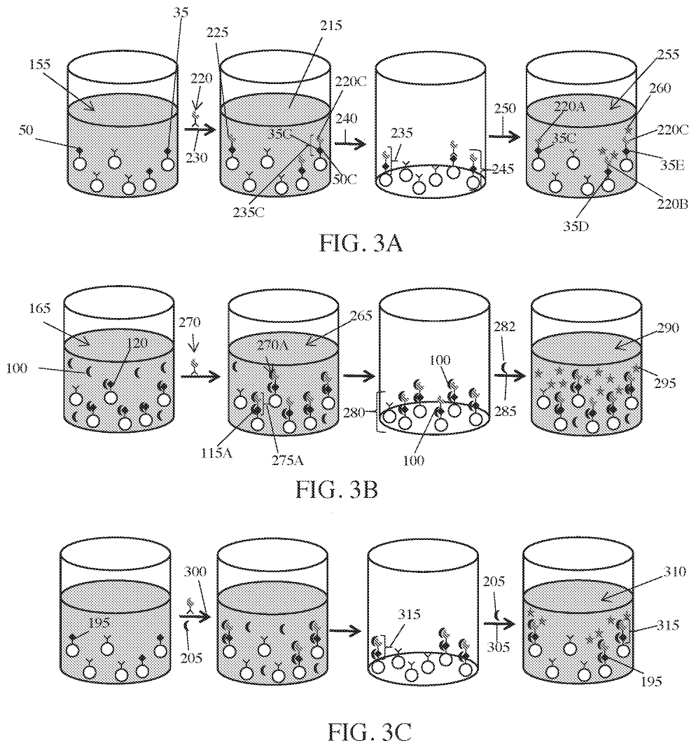

Additional details of the methods will now be described in connection with the figures. As described herein, a stabilizing agent may be used in one or more assay steps to reduce and/or prevent signal decay. In some embodiments, as illustrated in FIG. 1-FIG. 3, the stabilizing agent may be used to reduce and/or prevent dissociation between components (e.g., analyte molecules, detection molecules, capture components, etc.) during one or more assay steps. For instance, the stabilizing agent may be used to reduce and/or prevent dissociation between components that are reversible associated, e.g., via an affinity interaction. As will be known to those of ordinary skill in the art, some reversible affinity interactions, such as those between certain analyte molecules (e.g., antigen) and detection molecules (e.g., capture object, antibody), are governed by thermodynamics. Accordingly, at some point during an assay step, some dissociation may occur. This may result in a reduced number of analyte molecules being detected than are actually present as illustrated in FIG. 1A, FIG. 2A, and FIG. 3A, as described in more detail herein.

In general, the dissociation constant of and/or dissociation rate between a pair of reversibly associated molecules (e.g., antibody-antigen pair), may be altered (e.g., adversely) by certain assay conditions (e.g., ion concentration, pH, temperature steps, solvent) and/or assay steps, such as chemical and/or physical manipulation of the molecules after association (e.g., washing, desolvation, pelleting, drying, exposure to certain fluids, exposure to reagents). Moreover, for certain molecules and/or under certain assay conditions, the amount of dissociation may increase and/or the total number of reversibly associated molecules may decrease as a function of time. Thus, in some existing detection assays, assay conditions, assay steps, and the time between initial association and detection, amongst other factors, may affect the degree to which dissociation alters detection and quantification of analyte molecules. Stabilizing agents may be used, as described herein, to reduce the effects of dissociation processes.

A non-limiting example of dissociation in a detection assay is shown in FIG. 1A. In some embodiments, an existing detection assay 10 may utilize detection molecules, such as capture objects 15, that reversibly associate with analyte molecules, for example, to aid in the detection of the analyte molecules. In certain embodiments, capture objects 15 may comprise a least one capture component 20 and a support 25. Capture component 20 may be selected to reversibly associate with the analyte molecule via an affinity interaction. In some cases, capture component 20 may be selected to undergo a specific affinity interaction (e.g., specific binding) with the analyte molecule. In certain embodiments, support 25 may serve to immobilize, allow for facile isolation, and/or allow for facile manipulation of capture component 20 and associated molecules. For instance, support 25 may be a solid capture object, such as in the form of a bead (e.g., magnetic bead) as illustrated that is attached (e.g., covalently, noncovalently) to capture component 20. The bead may be easily separated from other assay component using simple separation techniques based on, e.g., magnetic force, gravitational force, density, centrifugal force, filtration, and/or evaporation. Other types of supports may be used, for example, a planar surface.

In some embodiments, capture objects 15 may be exposed to a sample 30 (e.g., fluid sample) comprising analyte molecules 35 as indicated by arrow 40. In certain embodiments, sample 30 may comprise other molecules 45 in addition to the analyte molecules 35 as illustrated in FIG. 1A. In certain embodiments, sample 30 may be combined with a solution 55 comprising capture objects 15 and a fluid (e.g., liquid) 32 as illustrated in FIG. 1A. In other embodiments, capture objects 15 may be added to sample 30. Upon exposure of the analyte molecules to the detection molecules, at least some of analyte molecules 35 may reversibly associate with capture components 20 to form associations 50. For example, analyte molecules 35 and capture component 20 may associated via an affinity interaction, such as an antibody-antigen interaction. In some such cases, capture component 20 may be an antigen and analyte molecule 35 may be an antibody or capture component 20 may be an antibody and analyte molecule 35 may be an antigen. In some embodiments, after association of at least some of analyte molecules 35 with capture objects 15, solution 55 may be allowed to remain for a period of time prior to the next assay step and/or signal detection as indicated by arrow 60. During this interim time period, at least some of associations 50 may dissociate. For instance, as illustrated in FIG. 1A, association 50A may dissociate.

In certain embodiments, after dissociation of at least a portion of associations 50, the next assay step and/or detection may occur as indicated by arrow 65. For example, the next assay step may comprise isolation and/or desolvation of (e.g., removal of liquid from) associations 50. In some such embodiments, at least a portion of (e.g., greater than or equal to 95%, greater than or equal to 98%, substantially all of) liquid 32 may be separated from associations 50 and optionally removed. For instance, liquid 32 may be separated from capture objects 15 by application of force (e.g., centrifugal force, gravitational force, magnetic force). In some such cases, the application of force (e.g., centrifugal force) may result in the formation of a composition comprising separated liquid and a separated assay mixture (e.g., pellet) 70, and the liquid may be removed (e.g., by filtration, decanting, and aspiration). Assay mixture 70 may comprise capture objects 15, associations 50, and optionally at least some molecules 45. In other cases, at least a portion of liquid 32 may be separated from capture objects 15 and/or associations 50 without the application of force and/or the formation of a pellet.

In some embodiments, the separated liquid may comprise at least some (e.g., substantially all) of the free analyte molecules that dissociated from capture objects 15. Removal of the separated liquid may result in the removal of at least some free analyte molecules thereby preventing re-association of the capture objects and the free analyte molecules. Regardless of the next assay step, the dissociation that occurred in the interim period between assay steps may result in a reduced signal and/or a reduced number of analyte molecules being detected.

In some embodiments, the stabilizing agents, described herein, may be used to reduce and/or prevent signal loss due to dissociation, such as that illustrated in FIG. 1A. A non-limiting example of a detection assay using a stabilizing agents to reduce and/or prevent dissociation is shown in FIG. 1B. Detection assay 80 may be substantially the same as detection assay 10, except for the use of a stabilizing agent. For example, capture objects 85 may comprise a capture component 90 and a support 95 (e.g., bead). Capture component 90 may also be selected to reversibly associate with the analyte molecule via an affinity interaction. Support 95 may also serve to immobilize, allow for facile isolation of, and/or allow for facile manipulation of capture component 90 and associated molecules. In some embodiments, in the presence of stabilizing agent 100, capture objects 85 may be exposed to a sample 105 (e.g., fluid sample) comprising analyte molecules 115 as indicated by arrow 110. In certain embodiments, sample 105 may be combined with a solution 125 comprising capture objects 85, stabilizing agent 100, and a fluid (e.g., liquid) 108 as illustrated in FIG. 1B. In other embodiments, the stabilizing agent may be added after (e.g., immediately after, within 30 seconds) exposure of the capture objects to the analyte molecules. Upon exposure of the capture objects to the analyte molecules, at least some of analyte molecules 115 may reversibly associate with capture components 90 to form associations 120. Stabilizing agent 100 may associate with associations 120 and alter (e.g., reduce) the rate of dissociation between and/or dissociation constant of the capture object 85 and analyte molecule 115. After association of at least some of analyte molecules 115 with capture objects 85, solution 125 may be allowed to remain for a period of time prior to the next assay step and/or signal detection as indicated by arrow 130. During this interim time period, stabilizing agent 100 may reduce and/or prevent at least some (e.g., substantially all) of associations 120 from dissociating as illustrated by arrow 135. In certain embodiments, the stabilizing agent may serve to reduce and/or prevent dissociation until the next assay step and/or detection.

For instance, in some embodiments, the next assay step may comprise isolation and/or desolvation of associations 120. In some such embodiments, at least a portion of (e.g., greater than or equal to 95%, substantially all of) liquid 108 may be separated from associations 120 and optionally removed as described above with respect to FIG. 1A. For example, separation of the liquid may result in the formation of a composition comprising the separated liquid and an assay mixture (e.g., pellet) 140. Assay mixture 140 may comprise capture objects 85 and stabilizing agents 100 associated with associations 120. In certain embodiments, the separated liquid from FIG. 1B may comprise substantially less free analyte molecules than the separated liquid from FIG. 1A. For instance, separated liquid from FIG. 1B may comprise little or no free analyte molecules due to dissociation. In such cases, removal of the liquid may result in minimal or no signal decay unlike existing assay 10. In some embodiments, the reduced dissociation associated with assay 80 may result in more accurate determination and quantification of analyte molecules.

As described above, existing detection assay 10 may comprise separating and/or removing at least a portion of a liquid from assay mixture 70. In some embodiments, as illustrated in FIG. 2A, a relatively large percentage of the liquid may be removed from the assay mixture, such that at least a portion of associations 50 are desolvated. It should be understood that the last container in FIG. 1A is the first container in FIG. 2A. In some such cases, after removal of the liquid, assay mixture 70 may be allowed to remain for a period of time prior to the next assay step and/or signal detection as indicated by arrow 150. For instance, assay mixture 70 may be stored for a period of time (e.g., at least 1 minute). During storage, at least some of associations 50 may dissociate. For instance, as illustrated in 2A, association 50B may dissociate. In some instances, the dissociations may occur in the time period between formation of the desolvated assay mixture and the next assay step (e.g., washing) and/or signal measurement. In some embodiments, the next assay step may comprise suspending assay mixture 70 in a fluid to form a solution 155 as indicated by arrow 158. Suspension of assay mixture 70 in the fluid may allow analyte molecule 35B to freely diffuse or otherwise move away from capture object 15B greatly reducing the likelihood of re-association between the capture object and the analyte molecule. In certain embodiments, analyte molecule 35B may not re-associate with a capture object prior to subsequent removal of the fluid and signal measurement. In such cases, the dissociation that occurred in the desolvated assay mixture may result in a reduced signal and/or a reduced number of analyte molecules being detected.

In some embodiments, the stabilizing agents, described herein, can be used to prevent dissociation in desolvated assay mixtures. For example, as described above, detection assay 80 may comprise separating and/or removing liquid from assay mixture 140. As illustrated in FIG. 1B and FIG. 2B, a relatively large percentage of the liquid may be removed from the assay mixture, such that at least a portion of associations 120 are desolvated. Stabilizing agents 100 may be associated with associations 120 in the desolvated state. In some such cases, after removal of the liquid, assay mixture 140 may be allowed to remain for a period of time prior to the next assay step and/or signal detection as indicated by arrow 160. The stabilizing agent may serve to prevent dissociation between capture objects 85 and analyte molecules 115 by maintaining a similar hydrogen bond network as the hydrogen bond network that was provided by the water prior to desolvation. In such cases, the stabilizing agent may allow a capture object and analyte molecule to maintain a similar structure and association while in the desolvated state as was present in solution. It should be understood that the last container in FIG. 1B is the first container in FIG. 2B. Assay mixture 140 may be suspended in a fluid to form a solution 165, as described above with respect to FIG. 2A, as indicated by arrow 170. Optionally, the fluid used for suspension may comprise additional stabilizing agent 102. In some embodiments, stabilizing agents 100 and/or optionally stabilizing agents 102 may reduce and/or prevent dissociation of at least some (e.g., substantially all) of associations 120. The reduced dissociation may result in more accurate determination and quantification of analyte molecules.

In some embodiments, the stabilizing agents, described herein, can be used to prevent dissociation during suspension of a desolvated assay mixtures that does not comprise a stabilizing agent as illustrated in FIG. 2C. FIG. 2C shows an assay mixture 180 substantially identical to assay mixture 70 of FIG. 2A. At least some of associations 185 in assay mixture 180 may also dissociate in the time period prior to the next assay step as illustrated by the dissociation between capture object 190A and analyte molecule 195A. In some embodiments, assay mixture 150 may be suspended in a solution 200 comprising a stabilizing agent 205, as indicated by arrow 210. The stabilizing agent may serve to promote association, such that that dissociated analyte molecules may readily re-associate with capture objects prior to the next assay step and/or signal measurement. For example, analyte molecule 195A that dissociated from capture object 190A in the time period prior to suspension may re-associate after suspension in solution 200.

In some embodiments, as illustrated in FIG. 3, the stabilizing agent may be used to reduce and/or prevent reduction of a measurable property of the detection molecules (e.g., enzymatic activity) and/or denaturation during one or more assay steps. As will be known to one of ordinary skill in the art, a measurable property of the detection molecules, such as enzyme activity, and the structural configuration of certain molecules (e.g., proteins) may be altered (e.g., adversely) by certain assay conditions and/or assay steps. For instance, certain ion concentrations, pHs, temperatures, and/or solvents may reduce the activity of an enzyme and result in denaturation of a protein. As another example, physical manipulation (e.g., washing, desolvation, agitation, pelleting) of certain detection molecules (e.g., enzymes, proteins) can negatively affect the ability of the detection molecules to association with analyte molecules (e.g., due to denaturation) and/or other detection molecules and/or the measurable property of the detection molecule. Thus, in some existing detection assays, certain assay conditions and assay steps, amongst other factors, may affect the degree to which reduction a measurable property of the detection molecules and/or denaturation alters detection and quantification of analyte molecules. Stabilizing agents may be used, as described herein, to reduce the effects of these processes.

A non-limiting example of reduction of a measurable property of the detection molecules and denaturation in a detection assay is shown in FIG. 3A. As described above, existing detection assay 10 may comprise suspension of assay mixture 70 in a fluid to form a solution 155. In some embodiments, production of a signal indicative of the presence, absence, and/or quantity of the analyte molecule may comprise exposing associations 50 to a solution 215 comprising binding ligand 220 have a measurable property (e.g., conductivity, spectroscopic emission, spectroscopic absorbance, fragmentation pattern, electrical potential, nucleic acid tags that can be amplified) as illustrated in FIG. 3A. In certain embodiments, binding ligand 220 comprises a component 225 that facilitates indirect detection of the analyte molecule, for example, by converting a precursor labeling agent into a labeling agent (e.g., an agent that is detected in an assay). For instance, binding ligand may comprise a component that reversibly and specifically associates with the analyte molecule (e.g., antibody) and an enzyme that converts precursor labeling agent (e.g., enzymatic substrate) into a labeling agent (e.g., enzymatic product). Upon exposure of analyte molecule 35 with binding ligand 220 as indicated by arrow 30, at least some analyte molecules 35 may associate with binding ligand 220. For instance, analyte molecule 35C in association 50C may reversibly associate with the binding ligand 220C to form a complex 235C. In some such embodiments, analyte molecules 35 and binding ligand 220 may associated via an affinity interaction, such as an antibody-antigen interaction. In some embodiments, after association between analyte molecules 35 and binding ligand 220, solution 215 may be allowed to remain for a period of time prior to the next assay step (e.g., detection). In some embodiments, the next assay step may involve separating and/or removing at least a portion of the fluid in solution 215. For instance, as indicated by arrow 240, detection assay 10 may comprise removing fluid from assay mixture 245 comprising complexes 235. In some embodiments, as illustrated in FIG. 3A, a relatively large percentage of the fluid may be removed from the assay mixture, such that at least a portion of complex 235 are desolvated.

In some embodiments, after removal of the liquid, the desolvated assay mixture may be allowed to remain for a period of time prior to the next assay step and/or signal detection. For instance, assay mixture 245 may be stored for a period of time (e.g., at least 1 minute). During storage, at least a portion of binding ligands 220 may denature and/or a measurable property (e.g., enzymatic activity) of binding ligand 220 may be altered, such that upon rehydration (e.g., suspension in a fluid) the structural configuration of the binding ligand is adversely altered and/or the measurable property is reduced. In certain embodiments, at least some of associations between the binding ligand and the analyte molecule and/or the analyte molecule and the capture object may dissociate. In some embodiments, the next assay step may comprise suspending assay mixture 235 in a fluid to form a solution 255 as indicated by arrow 250. At least a portion of the binding ligands in solution 255 may be denatured. For example, binding ligand 220A may be denatured in the solution. In certain embodiments, the measurable property of binding ligand 220C may be reduced. In embodiments in which dissociation occurs, the suspension of assay mixture may allow binding ligand to freely diffuse or otherwise move away from analyte molecules greatly reducing the likelihood of re-association between the binding ligand and the analyte molecule. In certain cases, re-association may not occur prior to subsequent removal of the fluid and/or signal measurement.

In some embodiments, the reduction of a measurable property, denaturation, and/or optional dissociation that occurred in the desolvated assay mixture may result in a reduced signal and/or a reduced number of analyte molecules being detected than are actually present. As illustrated in FIG. 3A, solution 255 may comprise three analyte molecules (e.g., 35C, 35D, 35E). Analyte molecule 35C may be associated with a denatured binding ligand 220A that cannot produce a signal. Analyte molecule 35D may be associated with a binding ligand 220B that is not denatured. The measurable property of binding ligand 220B may not be altered, such that the magnitude of the measurable property is the substantially the same as the magnitude of the measurable property prior to utilization in the assay and/or prior to the removal step. For instance, in embodiments in which the measurable property is enzymatic activity of an enzyme component, the activity of the enzyme is the substantially the same as the activity of the enzyme prior to utilization in the assay and/or prior to the removal step. In some such embodiments, the enzyme may convert precursor labeling agent into labeling agent 260 to produce a signal indicative of the analyte molecule. Conversely, analyte molecule 35E may be associated with binding ligand 220C that has measurable property, which is reduced. In some such cases, the magnitude of the measurable property is less than the magnitude of the measurable property prior to utilization in the assay and/or prior to the removal step. For instance, in embodiments in which the measurable property is enzymatic activity of an enzyme component, the activity of the enzyme is the substantially less than the activity of the enzyme prior to utilization in the assay and/or prior to the removal step. In some such embodiments, the rate of conversion of precursor labeling agent into labeling agent 260 may be reduced relative the rate of conversion of the enzyme in binding ligand 220B. For example, during the same time interval, binding ligand 220C may produce one labeling agent and binding ligand 220B may produce two labeling agents as illustrated in FIG. 3A. In some embodiments, the signal indicative of the analyte molecules may be reduced by at least about 10% (e.g., at least about 15%, at least about 20%, at least about 30%, at least about 40%, at least about 50%, at least about 60%, at least about 70%, at least about 80%, at least about 90%) due to the denaturation and/or reduction of the measurable property of the binding ligand. In some embodiments, the absence of stabilizing agent in detection assay 10 may result in an overall signal decay of at least about 50% (e.g., at least about 60%, at least about 70%, at least about 80%, at least about 90%, 100%) as illustrated by comparison of labeling agent in FIG. 3A and labeling agent in FIG. 3B.

In some embodiments, the stabilizing agents, described herein, can be used to prevent reduction of a measurable property of the detection molecules and/or denaturation. For example, as described above, detection assay 80 may comprise suspension of assay mixture 140 in a fluid to form a solution 165 containing stabilization agents 100. In some embodiments, production of a signal indicative of the presence, absence, and/or quantity of the analyte molecule may comprise exposing associations 120 to binding ligand 270, thereby forming solution 265. Binding ligand 270 may have a measurable property (e.g., conductivity, spectroscopic emission, spectroscopic absorbance, fragmentation pattern, electrical potential, nucleic acid tags that can be amplified) as described above with respect to FIG. 3A and illustrated in FIG. 3B. Upon exposure of the binding ligands to the analyte molecules, at least some of analyte molecules 115 may associate with binding ligands 270. For instance, analyte molecule 115A in an association may reversibly associate with the binding ligand 270A to form a complex 275A, as described herein. In some embodiments, at least a portion of the fluid in solution 265 may be removed to form a desolvated assay mixture as described above with respect to FIG. 3A. In some embodiments, after removal of the liquid, the desolvated assay mixture may be allowed to remain for a period of time prior to the next assay step and/or signal detection. For instance, assay mixture 280 may be stored for a period of time (e.g., at least 1 minute). Stabilizing agents 100 may be associated with the binding ligand and/or associations. The stabilizing agent may serve to prevent denaturation, dissociation from the analyte, and/or reduction of a measurable property of the binding ligands. In certain embodiments, the stabilizing agent may serve to prevent dissociation between binding ligands (e.g., antibody) and analyte molecules (e.g., protein) as well as capture objects and analyte molecules. Assay mixture 280 may be suspended in a fluid, as described above with respect to FIG. 3A, as indicated by arrow 285 as shown in FIG. 3B. Optionally, the fluid may comprise additional stabilizing agent 282. In some embodiments, stabilizing agents 100 and/or optional stabilizing agents 282 may reduce denaturation, reduction of a measurable property, and/or optionally dissociation.

In some embodiments, utilization of a stabilizing agents in one or more assay steps may result in an increased signal. In certain embodiments, the increase in signal may be due to an increase in the capture and/or retention of analyte molecules due to a reduction in dissociation. For instance, as illustrated in FIG. 3B, solution 290 has more associations between capture objects and analyte molecules, as well as between analyte molecules, and binding ligands than solution 255 in detection assay 10 of FIG. 3A. Accordingly, solution 290 in FIG. 3B has more labeling agents 295 than solution 255 in FIG. 3A. In some embodiments, the increase in signal may be due to the reduction in dissociation of the associations necessary for detection.

In certain embodiments, the increase in signal may be due to preservation of a measurable property of the detection molecules as illustrated in FIG. 3C. In some embodiments, the signal of a measurable property may be maintained by exposing the assay mixture to a stabilizing agent 205 during the interim period between an assay step and detection. FIG. 3C depicts the same assay as FIG. 3A, except the assay mixture is exposed to a stabilizing agent prior to liquid removal as indicated by arrow 300 and optionally during suspension as indicated by arrow 305. The stabilizing agent may serve to prevent alteration of the measurable property (e.g., enzymatic activity) that may result in signal loss. Though solution 310 in FIG. 3C has the same number of analyte molecules 195 and complexes 315 as solution 255 in FIG. 3A, solution 310 produces a greater signal than solution 255 due to the maintenance of the measurable property (e.g., enzyme activity) as well as the lack of denaturation.

It should be understood that FIG. 1-FIG. 3 are for illustrative purposes and that a suitable detection assay may comprise some, all, or none of the assay steps and/or components (e.g., binding ligands, capture objects) illustrated in the figures. Moreover, it should be understood that a stabilizing agent need not be used in every assay step, as illustrated in certain figures, to achieve desirable results (e.g., increase signal, reduction in dissociation, reduction in denaturation, maintenance of a measurable property).

As described herein, a stabilizing agent can be used to reduce and/or prevent signal decay. In some embodiments, the stabilizing agent may be a polyol. In some such embodiments, the stabilizing agent may comprise greater than or equal to about 2, greater than or equal to about 3, greater than or equal to about 4, greater than or equal to about 5, greater than or equal to about 6, greater than or equal to about 7, greater than or equal to about 8, greater than or equal to about 9, greater than or equal to about 10, greater than or equal to about 11, greater than or equal to about 12, or greater than or equal to about 13 hydroxyl groups. In some instances, the stabilizing agent may comprise less than or equal to about 14, less than or equal to about 13, less than or equal to about 12, less than or equal to about 11, less than or equal to about 10, less than or equal to about 9, less than or equal to about 8, less than or equal to about 7, less than or equal to about 6, less than or equal to about 5, or less than or equal to about 4 hydroxyl groups. All combination of the above-referenced ranges are possible (e.g., greater than or equal to about 2 and less than or equal to about 14 hydroxyl groups, greater than or equal to about 4 and less than or equal to about 12 hydroxyl groups). In some embodiments, the stabilizing agent may comprise a polyol as a repeat unit with the above-referenced range of hydroxyl groups. For example, the stabilizing agent may be a polysaccharide.

In some embodiments, the stabilizing agent may be a carbohydrate. In certain embodiments, the stabilizing agent may be a small molecule carbohydrate, such as sucrose, trehalose, and combinations thereof. In some embodiments, the stabilizing agent may be a small molecule polyol. In some embodiments, the stabilizing agent may be a kosmotrope (e.g., non-ionic kosmotrope, zwitterionic kosmotrope) and/or an osmolyte. Non-limiting example of suitable stabilizing agents include glycerol, ethylene glycol, propylene glycol, sorbitol, mannitol, ectoine ((S)-2-methyl-3,4,5,6-tetrahydropyrimidine-4-carboxylic acid), proline, Glycine betaine, glucose, trimethylamine-N-oxide, glycine, N-methylglycine (sarcosine), N,N-dimethylglycine, N,N,N-trimethylglycine (betaine), dimethylsulfoniopropionate, glycerophosphorylcholine, myo-inositol, and taurine.

In some embodiments, the beneficial properties of the stabilizing agent may depend, at least in part, on the weight percentage of the stabilizing agent in the assay composition (e.g., assay mixture, solution). For instance, in some embodiments, the weight percentage of the stabilizing agent in the assay composition (e.g., solution, fluid, mixture, pellet) may be greater than or equal to about 0.5 wt. %, greater than or equal to about 1 wt. %, greater than or equal to about 2 wt. %, greater than or equal to about 4 wt. %, greater than or equal to about 5 wt. %, greater than or equal to about 6 wt. %, greater than or equal to about 8 wt. %, greater than or equal to about 10 wt. %, greater than or equal to about 12 wt. %, greater than or equal to about 14 wt. %, greater than or equal to about 15 wt. %, greater than or equal to about 16 wt. %, greater than or equal to about 18 wt. %, greater than or equal to about 20 wt. %, greater than or equal to about 22 wt. %, greater than or equal to about 24 wt. %, greater than or equal to about 25 wt. %, greater than or equal to about 26 wt. %, or greater than or equal to about 28 wt. %. In some instances, the weight percentage of the stabilizing agent in the assay composition (e.g., solution, fluid, mixture, pellet) may be less than or equal to about 30 wt. %, less than or equal to about 28 wt. %, less than or equal to about 26 wt. %, less than or equal to about 25 wt. %, less than or equal to about 24 wt. %, less than or equal to about 22 wt. %, less than or equal to about 20 wt. %, less than or equal to about 18 wt. %, less than or equal to about 16 wt. %, less than or equal to about 15 wt. %, less than or equal to about 14 wt. %, less than or equal to about 12 wt. %, less than or equal to about 10 wt. %, less than or equal to about 8 wt. %, less than or equal to about 6 wt. %, less than or equal to about 5 wt. %, or less than or equal to about 3 wt. %. All combinations of the above-referenced ranges are possible (e.g., greater than or equal to about 1 wt. % and less than or equal to about 30 wt. %, greater than or equal to about 10 wt. % and less than or equal to about 30 wt. %).

As noted above, a method may comprise using a stabilizing agent prior to, during, and/or after one or more steps of a detection assay. In general, the stabilizing agent may be used prior to, during, and/or after any suitable assay step. For instance, the stabilizing agent may be used prior to removal of at least a portion (e.g., substantially all) of a fluid from an assay mixture comprising associated molecules (e.g., capture objects, binding ligands, analyte molecules). In some instances, the stabilizing agent may be used prior to and/or during storage of an assay mixture comprising associated molecules (e.g., capture objects, binding ligands, analyte molecules). In certain embodiments, the stabilizing agent may be used prior to (e.g., immediately prior to), during, and/or after (e.g., immediately after) addition of a binding ligand. In certain cases, the stabilizing agent may be used prior to and/or during signal measurement. In some embodiments, the stabilizing agent may be used in substantially all assay steps. Non-limiting examples of assay steps in which a stabilizing agent may be used prior to, during, and/or after include washing, desolvation, agitation, suspension, isolation, incubation, storage, measurement, dilution, mixing, and drying steps.

In some embodiments, the stabilizing agent may be used in a relatively large percentage (e.g., greater than or equal to about 50%, substantially all) of the steps in a detection assay. For instance, the stabilizing agent may be a component in an assay buffer that is used in at least some (e.g., substantially all) of the assay steps that utilize a buffer. As another example, the stabilizing agent may be a component of the wash solution (e.g., wash buffer) for the assay. In some such cases, the stabilizing agent may be used prior to and/or after a relatively large percentage (e.g., greater than or equal to about 50%, substantially all) of steps in a detection assay. In other embodiments, the stabilizing agent may be used in relatively small percentage of assay steps. For instance, the stabilizing agent may be use in less than or equal to about 4 assay steps (e.g., less than or equal to about 2 assay steps, a single assay step). Regardless of the number of steps that utilize the stabilizing agent, the stabilizing agent may prevent and/or reduce signal decay.

In some embodiments, the stabilizing agent may be used to reduce and/or prevent signal loss during storage of an assay mixture that comprises, for example, associations (e.g., capture objects, binding ligands, analyte molecules). In general, the assay mixture may be stored for any suitable amount of time with minimal or no loss in signal due to, e.g., dissociation, denaturation, and/or reduction in a measurable property. For instance, an assay mixture may be stored for greater than or equal to about 5 seconds, greater than or equal to about 15 seconds, greater than or equal to about 30 seconds, greater than or equal to about 45 seconds, greater than or equal to about 1 minute, greater than or equal to about 2 minutes, greater than or equal to about 4 minutes, greater than or equal to about 5 minutes, greater than or equal to about 8 minutes, greater than or equal to about 10 minutes, greater than or equal to about 30 minutes, greater than or equal to about 1 hour, greater than or equal to about 2 hours, greater than or equal to about 6 hours, greater than or equal to about 12 hours, greater than or equal to about 18 hours, greater than or equal to about 1 day, greater than or equal to about 2 days, greater than or equal to about 3 days, greater than or equal to about 5 days minutes, greater than or equal to about 1 week, greater than or equal to about 2 weeks, greater than or equal to about 3 weeks, or greater than or equal to about 1 month with minimal or no loss in signal due to, e.g., dissociation, denaturation, and/or reduction in a measurable property. In some instances, an assay mixture may be stored for less than or equal to about 3 months, less than or equal to about 2 months, less than or equal to about 1 month, less than or equal to about 3 weeks, less than or equal to about 2 weeks, less than or equal to about 7 days, less than or equal to about 5 days, less than or equal to about 3 days, less than or equal to about 2 days, less than or equal to about 24 hours, less than or equal to about 18 hours, less than or equal to about 12 hours, or less than or equal to about 6 hours with minimal or no loss in signal due to, e.g., dissociation, denaturation, and/or reduction in a measurable property. All combination of the above-referenced ranges are possible (e.g., greater than or equal to about 5 seconds and less than or equal to about 3 months, greater than or equal to about 12 hours and less than or equal to about 3 months). In general, any suitable storage conditions may be used. In some embodiments, the storage condition may be an assay condition. In certain embodiments, storage may occur at a certain temperature (e.g., between about −85° C. and about 25° C., between about −22° C. and about 25° C., between about −6° C. and about 25° C., between about 2° C. and about 25° C., between about 20° C. and about 25° C., between about 2° C. and about 8° C., between about −85° C. and about 20° C., between about −85° C. and about 8° C., between about −85° C. and about −2° C., between about −85° C. and about −18° C., between about −6° C. and about −2° C., between about −18° C. and about −22° C., and between about −75° C. and about −85° C.), at a certain pressure (e.g., about atmospheric pressure, less than atmospheric pressure, including vacuum), in a solvated state (e.g., in solution) and/or in a desolvated state (e.g., substantially dry).

As used herein, the terms “storage” and “stored” as well as other grammatical variations have their ordinary meaning in the art and may refer to the interim period between the end of one assay step and the beginning of the next assay step. In some embodiments, storage may occur during the operation of an assay due to, e.g., the time required for the set-up of an assay step and/or the concurrent analysis of multiple replicates and/or samples. For instance, in embodiments in which multiple samples are assayed concurrently, the individual detection of one sample may result in storage of another sample. In some embodiments, storage may occur due to, e.g., temporary cessation of the assay. For instance, an assay may be temporarily suspended after formation of a certain assay mixture to allow for analysis at a different time and/or location. For instance, an assay may be manufactured at a manufacturing facility and stored before shipping to a customer and its ultimate use.

In some embodiments, the stabilizing agent may be used to reduce and/or prevent signal loss during removal of a fluid from an assay mixture comprising associations (e.g., capture objects, binding ligands, analyte molecules). In general, any suitable method may be used to remove at least a portion (e.g., substantially all) of the fluid from the assay mixture. Non-limiting examples include aspiration, vacuum filtration, exposure to reduced pressure below the vapor pressure of the fluid in the presence or absence of a desiccant, evaporation, drying in a stream of air, and/or heating. In some embodiments, removal of the fluid may be result in desolvation of the assay mixture. In some such cases, the assay mixture may be substantially dry. For instance, the desolvated assay mixture may comprise less than or equal to about 5 wt. %, less than or equal to about 4 wt. %, less than or equal to about 3 wt. %, less than or equal to about 2 wt. %, less than or equal to about 1 wt. %, less than or equal to about 0.5 wt. %, less than or equal to about 0.1 wt. %, less than or equal to about 0.05 wt. %, or less than or equal to about 0.001 wt. % of a fluid. In some embodiments, the assay mixture may be desolvated prior to certain assay steps (e.g., washing, storage).

In some embodiments, the stabilizing agent may reduce and/or prevent dissociation. For instance, less than or equal to about 20% (e.g., less than or equal to about 18%, less than or equal to about 15%, less than or equal to about 12%, less than or equal to about 10%, less than or equal to about 8%, less than or equal to about 5%, less than or equal to about 3%, less than or equal to about 2%, less than or equal to about 1%, less than or equal to about 0.8%, less than or equal to about 0.5%, less than or equal to about 0.3%, less than or equal to about 0.1%, less than or equal to about 0.05%, less than or equal to about 0.01%) of analyte molecules and/or detection molecules (e.g., binding ligands, capture objects) may dissociate during one or more assay steps and/or the entire assay. In some embodiments, the percent dissociation may be determined using surface plasmon resonance and/or interferometrometric techniques.

In some embodiments, the stabilizing agent may reduce the dissociation constant and/or dissociation rate. For instance, the stabilizing agent may reduce the dissociation constant and/or rate by greater than or equal to about 0.5% (e.g., greater than or equal to about 1%, greater than or equal to about 2%, greater than or equal to about 3%, greater than or equal to about 5%, greater than or equal to about 8%, greater than or equal to about 10%, greater than or equal to about 15%, greater than or equal to about 20%). The dissociation rate and/or constant may be determined using surface plasmon resonance and/or interferometrometric techniques.

In some embodiments, the stabilizing agent may reduce and/or prevent alterations in the structural configuration of a molecule (e.g., denaturation). For instance, less than or equal to about 20% (e.g., less than or equal to about 18%, less than or equal to about 15%, less than or equal to about 12%, less than or equal to about 10%, less than or equal to about 8%, less than or equal to about 5%, less than or equal to about 3%, less than or equal to about 2%, less than or equal to about 1%, less than or equal to about 0.8%, less than or equal to about 0.5%, less than or equal to about 0.3%, less than or equal to about 0.1%, less than or equal to about 0.05%, less than or equal to about 0.01%) of analyte molecules and/or detection molecules (e.g., binding ligands, capture objects) undergo a structural alteration (e.g., denaturation) during one or more assay steps and/or the entire assay. In some embodiments, the structural alteration of a molecule may be determined using NMR, circular dichroism, and/or light scattering.

In some embodiments, the stabilizing agent may reduce and/or prevent reduction in a measurable property (e.g., enzyme activity) of a binding ligand. For instance, a measurable property of a binding ligand may be reduced by less than or equal to about 20% (e.g., less than or equal to about 18%, less than or equal to about 15%, less than or equal to about 12%, less than or equal to about 10%, less than or equal to about 8%, less than or equal to about 5%, less than or equal to about 3%, less than or equal to about 2%, less than or equal to about 1%, less than or equal to about 0.8%, less than or equal to about 0.5%, less than or equal to about 0.3%, less than or equal to about 0.1%, less than or equal to about 0.05%, less than or equal to about 0.01%) during one or more assay steps and/or the entire assay. The reduction may be measured by determining the measurable property before and after the assay step(s) and/or the entire assay. For example, enzyme activity may be determined using ELISA or Simoa techniques.

In some embodiments, the stabilizing agent may be used to maintain and/or improve a signal indicative of the analyte molecule during one or more assay steps and/or the entire assay. For instance, the stabilizing agent may be used to maintain the signal during storage. In some embodiments, the signal after storage for the times and under the conditions described herein may be greater than or equal to about 10%, may be greater than or equal to about 20%, may be greater than or equal to about 30%, may be greater than or equal to about 40%, may be greater than or equal to about 50%, may be greater than or equal to about 60%, may be greater than or equal to about 70%, greater than or equal to about 72%, greater than or equal to about 75%, greater than or equal to about 78%, greater than or equal to about 80%, greater than or equal to about 82%, greater than or equal to about 85%, greater than or equal to about 88%, greater than or equal to about 90%, greater than or equal to about 92%, greater than or equal to about 95%, greater than or equal to about 98%, greater than or equal to about 99%, or greater than or equal to about 100% of the signal prior to storage.

In some embodiments, the stabilizing agent may be used to reduce and/or prevent signal decay due to desolvation. In such cases, the signal after desolvation (e.g., in the solution after resuspension of the desolvated mixture) may be greater than or equal to about 10%, may be greater than or equal to about 20%, may be greater than or equal to about 30%, may be greater than or equal to about 40%, may be greater than or equal to about 50%, may be greater than or equal to about 60%, may be greater than or equal to about 70%, greater than or equal to about 72%, greater than or equal to about 75%, greater than or equal to about 78%, greater than or equal to about 80%, greater than or equal to about 82%, greater than or equal to about 85%, greater than or equal to about 88%, greater than or equal to about 90%, greater than or equal to about 92%, greater than or equal to about 95%, greater than or equal to about 98%, greater than or equal to about 99%, or greater than or equal to about 100% of the signal prior to desolvation.

In some embodiments, the stabilizing agent may be used to maintain and/or improve the biological activity of the analyte and/or detection molecule during one or more assay steps and/or the entire assay. For instance, the stabilizing agent may be used to maintain the biological activity of the detection molecule during storage. In some embodiments, the biological activity after storage for the times and under the conditions described herein may be greater than or equal to about 10%, may be greater than or equal to about 20%, may be greater than or equal to about 30%, may be greater than or equal to about 40%, may be greater than or equal to about 50%, may be greater than or equal to about 60%, may be greater than or equal to about 70%, greater than or equal to about 72%, greater than or equal to about 75%, greater than or equal to about 78%, greater than or equal to about 80%, greater than or equal to about 82%, greater than or equal to about 85%, greater than or equal to about 88%, greater than or equal to about 90%, greater than or equal to about 92%, greater than or equal to about 95%, greater than or equal to about 98%, greater than or equal to about 99%, or greater than or equal to about 100% of the biological activity prior to storage.

In some embodiments, the stabilizing agent may be used to maintain and/or improve the binding affinity (e.g., equilibrium dissociation constant, K_{D}) of the analyte for the detection molecule(s) during one or more assay steps and/or the entire assay. For instance, the stabilizing agent may be used to maintain the binding affinity of the detection molecule during storage. In some embodiments, the binding affinity after storage for the times and under the conditions described herein may be greater than or equal to about 10%, may be greater than or equal to about 20%, may be greater than or equal to about 30%, may be greater than or equal to about 40%, may be greater than or equal to about 50%, may be greater than or equal to about 60%, may be greater than or equal to about 70%, greater than or equal to about 72%, greater than or equal to about 75%, greater than or equal to about 78%, greater than or equal to about 80%, greater than or equal to about 82%, greater than or equal to about 85%, greater than or equal to about 88%, greater than or equal to about 90%, greater than or equal to about 92%, greater than or equal to about 95%, greater than or equal to about 98%, greater than or equal to about 99%, or greater than or equal to about 100% of the binding affinity prior to storage.

As used herein, the term “detection molecule” has its ordinary meaning in the art and may refer to a molecule that directly or indirectly associates with an analyte molecule and is used in the determination of the presence, absence, and/or quantity of the analyte molecule.

As used herein, the phrase “associated with” when used with respect to two or molecules (e.g., a capture object and an analyte molecule) refers to a direct or indirect link between the molecules formed via a chemical and/or biological interaction. In some embodiments, an association between molecules may occur via a biological binding event (i.e., between complementary pairs of biological molecules). For example, a molecule may include an entity such as biotin that specifically binds to a complementary entity, such as avidin or streptavidin, on another molecule. Other examples of biological molecules that may form biological bonds between pairs of biological molecules include, but are not limited to, proteins, nucleic acids, glycoproteins, carbohydrates, hormones, and the like. Specific examples include, but are not limited to, an antibody/peptide pair, an antibody/antigen pair, an antibody fragment/antigen pair, an antibody/antigen fragment pair, an antibody fragment/antigen fragment pair, an antibody/hapten pair, an enzyme/substrate pair, an enzyme/inhibitor pair, an enzyme/cofactor pair, a protein/substrate pair, a nucleic acid/nucleic acid pair, a protein/nucleic acid pair, a peptide/peptide pair, a protein/protein pair, a small molecule/protein pair, a glutathione/GST pair, an anti-GFP/GFP fusion protein pair, a Myc/Max pair, a maltose/maltose binding protein pair, a carbohydrate/protein pair, a carbohydrate derivative/protein pair, a metal binding tag/metal/chelate, a peptide tag/metal ion-metal chelate pair, a peptide/NTA pair, a lectin/carbohydrate pair, a receptor/hormone pair, a receptor/effector pair, a complementary nucleic acid/nucleic acid pair, a ligand/cell surface receptor pair, a virus/ligand pair, a Protein A/antibody pair, a Protein G/antibody pair, a Protein L/antibody pair, an Fc receptor/antibody pair, a biotin/avidin pair, a biotin/streptavidin pair, a drug/target pair, a zinc finger/nucleic acid pair, a small molecule/peptide pair, a small molecule/protein pair, a small molecule/target pair, a carbohydrate/protein pair such as maltose/MBP (maltose binding protein), a small molecule/target pair, or a metal ion/chelating agent pair. Biological interactions between a molecule and another molecule (e.g., binding partner) suitable for use in the embodiments described herein can be selected readily, by those of ordinary skill in the art, based upon the description herein as their function, examples of such biological interactions, and knowledge herein and in the art as to simple techniques for identifying suitable chemical interactions.

In some embodiments, molecule may associate via a chemical interaction, such as a chemical bond. In some embodiments, the association is formed via a non-covalent bond (e.g., hydrogen bonding, van der Waals interactions, hydrophobic interactions, magnetic interactions, electrostatic interactions, pi stacking, dipole-dipole interactions, ligand-receptor interaction). In some embodiments, the molecule and binding partner may comprise functional groups capable of forming such bonds. For example, a molecule may include at least one hydrogen atom capable of interacting with a pair of electrons on a hydrogen-bond acceptor of a binding partner to form the hydrogen bond. In some embodiments, a molecule and/or a binding partner may include an electron-rich or electron-poor moiety, such that it may form an electrostatic interaction with another of a binding partner and/or molecule, respectively. It should be understood that non-covalent bonds between components may be formed by any type of reactions, as known to those of ordinary skill in the art, using the appropriate functional groups to undergo such reactions. Chemical interactions suitable for use with various embodiments described herein can be selected readily by those of ordinary skill in the art, based upon the description herein.

As used herein, the term “small molecule” refers to molecules, whether naturally occurring or artificially created (e.g., via chemical synthesis) that have a relatively low molecular weight. Typically, a small molecule is an organic compound (i.e., it contains carbon). The small molecule may contain multiple carbon-carbon bonds, stereocenters, and other functional groups (e.g., amines, hydroxyl, carbonyls, and heterocyclic rings, etc.). In certain embodiments, the molecular weight of a small molecule is at most about 1,000 g/mol, at most about 900 g/mol, at most about 800 g/mol, at most about 700 g/mol, at most about 600 g/mol, at most about 500 g/mol, at most about 400 g/mol, at most about 300 g/mol, at most about 200 g/mol, or at most about 100 g/mol. In certain embodiments, the molecular weight of a small molecule is at least about 100 g/mol, at least about 200 g/mol, at least about 300 g/mol, at least about 400 g/mol, at least about 500 g/mol, at least about 600 g/mol, at least about 700 g/mol, at least about 800 g/mol, or at least about 900 g/mol, or at least about 1,000 g/mol. Combinations of the above ranges (e.g., at least about 200 g/mol and at most about 500 g/mol) are also possible.

As used herein, the term “mixture” has its ordinary meaning in the art and typically refers to a system that contains two or more chemical substances (e.g., detection molecules, analyte molecules) that are combined such that each substance retains its own chemical identity. The mixture may be homogenous (e.g., a solution) or heterogeneous (e.g., a suspension). In some embodiments, the mixture is homogenous (e.g., a solution). In certain embodiments, the mixture is heterogeneous (e.g., a suspension).

The methods and system, described herein, may be used to extend the dynamic range of a detection assay. The term, “dynamic range” is given its ordinary meaning in the art and refers to the range of the concentration of analyte molecules in a fluid sample that may be quantitated by a system or method without dilution or concentration of the sample or change in the assay conditions producing a similar result (e.g., concentration of reagents employed, etc.), and wherein the measured concentration of the analyte molecules may be substantially accurately determined. The concentration of analyte molecules in a fluid sample may be considered to be substantially accurately determined if the measured concentration of the analyte molecules in the fluid sample is within about 10% of the actual (e.g., true) concentration of the analyte molecules in the fluid sample. In certain embodiments, the measured concentration of the analyte molecules in the fluid sample is substantially accurately determined in embodiments where the measured concentration is within about 5%, within about 4%, within about 3%, within about 2%, within about 1%, within about 0.5%, within about 0.4%, within about 0.3%, within about 0.2%, or within about 0.1% of the actual concentration of the analyte molecules in the fluid sample. In some cases, the measure of the concentration determined differs from the true (e.g., actual) concentration by no greater than about 20%, no greater than about 15%, no greater than about 10%, no greater than about 5%, no greater than about 4%, no greater than about 3%, no greater than about 2%, no greater than about 1%, or no greater than about 0.5%. The accuracy of the assay method may be determined, in some embodiments, by determining the concentration of analyte molecules in a fluid sample of a known concentration using the selected assay method and comparing the measured concentration with the actual concentration.

In some embodiments, the concentration (e.g., unknown concentration) of analyte molecules in the fluid sample that may be substantially accurately determined is less than about 5000 fM (femtomolar), less than about 3000 fM, less than about 2000 fM, less than about 1000 fM, less than about 500 fM, less than about 300 fM, less than about 200 fM, less than about 100 fM, less than about 50 fM, less than about 25 fM, less than about 10 fM, less than about 5 fM, less than about 2 fM, less than about 1 fM, less than about 500 aM (attomolar), less than about 100 aM, less than about 10 aM, less than about 5 aM, less than about 1 aM, less than about 0.1 aM, less than about 500 zM (zeptomolar), less than about 100 zM, less than about 10 zM, less than about 5 zM, less than about 1 zM, less than about 0.1 zM, or less. In some cases, the limit of detection (e.g., the lowest concentration of an analyte molecule which may be determined in solution) is about 100 fM, about 50 fM, about 25 fM, about 10 fM, about 5 fM, about 2 fM, about 1 fM, about 500 aM (attomolar), about 100 aM, about 50 aM, about 10 aM, about 5 aM, about 1 aM, about 0.1 aM, about 500 zM (zeptomolar), about 100 zM, about 50 zM, about 10 zM, about 5 zM, about 1 zM, about 0.1 zM, or less. In some embodiments, the concentration of analyte molecules or particles in the fluid sample that may be substantially accurately determined is between about 5000 fM and about 0.1 fM, between about 3000 fM and about 0.1 fM, between about 1000 fM and about 0.1 fM, between about 1000 fM and about 0.1 zM, between about 100 fM and about 1 zM, between about 100 aM and about 0.1 zM, or less. The upper limit of detection (e.g., the upper concentration of an analyte molecule which may be determined in solution) is at least about 100 fM, at least about 1000 fM, at least about 10 uM (picomolar), at least about 100 uM, at least about 100 uM, at least about 10 nM (nanomolar), at least about 100 nM, at least about 1000 nM, at least about 10 uM, at least about 100 uM, at least about 1000 uM, at least about 10 mM, at least about 100 mM, at least about 1000 mM, or greater. In some embodiments, the concentration of analyte molecules or particles in the fluid sample determined is less than about 50×10⁻¹⁵ M, or less than about 40×10⁻¹⁵ M, or less than about 30×10⁻¹⁵ M, or less than about 20×10⁻¹⁵ M, or less than about 10×10⁻¹⁵ M, or less than about, or less than about 1×10⁻¹⁵ M.

### Exemplary Assay Methods and Systems

Those of ordinary skill in the art will be aware of a variety of assay methods and systems that may be used in connection with the methods of the present invention. Generally, the methods employed have low limits of detection and/or limits of quantification as compared to bulk analysis techniques (e.g., ELISA methods). The use of assay methods that have low limits of detection and/or limits of quantification allows for correlations to be made between the various parameters discussed above and a method of treatment and/or diagnostic indication that may otherwise not be determinable and/or apparent.

In some embodiments, the analyte molecule may be an enzyme. Non-limiting examples of enzymes include, an oxidoreductase, transferase, kinase, hydrolase, lyase, isomerase, ligase, and the like. Additional examples of enzymes include, but are not limited to, polymerases, cathepsins, calpains, amino-transferases such as, for example, AST and ALT, proteases such as, for example, caspases, nucleotide cyclases, transferases, lipases, enzymes associated with heart attacks, and the like. When a system/method of the present invention is used to detect the presence of viral or bacterial agents, appropriate target enzymes include viral or bacterial polymerases and other such enzymes, including viral or bacterial proteases, or the like.

In other embodiments, the analyte molecule may comprise an enzymatic component. For example, the analyte particle can be a cell having an enzyme or enzymatic component present on its extracellular surface. Alternatively, the analyte particle is a cell having no enzymatic component on its surface. Such a cell is typically identified using an indirect assaying method described below. Non-limiting example of enzymatic components are horseradish peroxidase, beta-galactosidase, and alkaline phosphatase.

In yet other embodiments, the analyte molecule may be a biomolecule. Non-limiting examples of biomolecules include hormones, antibodies, cytokines, proteins, nucleic acids, lipids, carbohydrates, lipids cellular membrane antigens and receptors (neural, hormonal, nutrient, and cell surface receptors) or their ligands, or combinations thereof. Non-limiting embodiments of proteins include peptides, polypeptides, protein fragments, protein complexes, fusion proteins, recombinant proteins, phosphoproteins, glycoproteins, lipoproteins, or the like. As will be appreciated by those in the art, there are a large number of possible proteinaceous analyte molecules that may be detected or evaluated for binding partners using the present invention. In addition to enzymes as discussed above, suitable protein analyte molecules include, but are not limited to, immunoglobulins, hormones, growth factors, cytokines (many of which serve as ligands for cellular receptors), cancer markers, neurological markers, inflammatory markers, markers of cardiovascular disease, etc. Non-limiting examples of biomolecules include PSA, beta-amyloid protein, tau protein, TNF-alpha, toxins from C. difficile, Aβ40, Aβ42, C-Peptide, Eotaxin, G-CSF, GM-CSF, IFNα, IFNγ, MCP-3, MIP-1β, pNF-heavy, NSE, PD-L1, PIGF, P-Tau 181, P-Tau 231, TDP43, TNFβ, UCH-L1, VEGF, GM-CSF, IL-1α, IL-1β, IL-2, IL-4, IL-3, IL-5, IL-6, IL-8, IL-10, IL-12p40/23, IL-12p70, IL-13, IL-15, IL-16, IL-17A, IL-17A/F, IL-17C, IL-17F, IL-18, IL-22, IL-23, IL-28A, IL-33, IL-36β, IP-10, MCP-1, NF-light (NFL), HIV p24, TRAIL, troponin-I, α-Synuclein, BDNF, CA 19-9, CA-125, cathepsin S, CEA, c-MET, CRP, CXCL13, GFAP, leptin, and LIF.

In certain embodiments, the analyte molecule may be a host-translationally modified protein (e.g., phosphorylation, methylation, glycosylation) and the capture component may be an antibody specific to a post-translational modification. Modified proteins may be captured with capture components comprising a multiplicity of specific antibodies and then the captured proteins may be further bound to a binding ligand comprising a secondary antibody with specificity to a post-translational modification. Alternatively, modified proteins may be captured with capture components comprising an antibody specific for a post-translational modification and then the captured proteins may be further bound to binding ligands comprising antibodies specific to each modified protein.

In another embodiment, the analyte molecule is a nucleic acid. A nucleic acid may be captured with a complementary nucleic acid fragment (e.g., an oligonucleotide) and then optionally subsequently labeled with a binding ligand comprising a different complementary oligonucleotide.

Suitable analyte molecules and particles include, but are not limited to small molecules (including organic compounds and inorganic compounds), environmental pollutants (including pesticides, insecticides, toxins, etc.), therapeutic molecules (including therapeutic and abused drugs, antibiotics, etc.), biomolecules (including hormones, cytokines, proteins, nucleic acids, lipids, carbohydrates, cellular membrane antigens and receptors (neural, hormonal, nutrient, and cell surface receptors) or their ligands, etc.), whole cells (including prokaryotic (such as pathogenic bacteria) and eukaryotic cells, including mammalian tumor cells), viruses (including retroviruses, herpesviruses, adenoviruses, lentiviruses, etc.), spores, etc.

The fluid sample containing or suspected of containing an analyte molecule may be derived from any suitable source. In some cases, the sample may comprise a liquid, fluent particulate solid, fluid suspension of solid particles, supercritical fluid, and/or gas. In some cases, the analyte molecule may be separated or purified from its source prior to determination; however, in certain embodiments, an untreated sample containing the analyte molecule may be tested directly. The source of the analyte molecule may be synthetic (e.g., produced in a laboratory), the environment (e.g., air, soil, etc.), a mammal, an animal, a plant, or any combination thereof. In a particular example, the source of an analyte molecule is a human bodily substance (e.g., blood, serum, plasma, urine, saliva, tissue, organ, stool, cerebral spinal fluid, or the like). The volume of the fluid sample analyzed may potentially be any amount within a wide range of volumes, depending on a number of factors such as, for example, the number of capture objects used/available, the number of locations us/available, etc.

In some cases, the fluid sample may be diluted prior to use in an assay. For example, in embodiments where the source of an analyte molecule is a human body fluid (e.g., blood, serum), the fluid may be diluted with an appropriate solvent (e.g., a buffer such as PBS buffer). A fluid sample may be diluted about 1-fold, about 2-fold, about 3-fold, about 4-fold, about 5-fold, about 6-fold, about 10-fold, or greater, prior to use. The sample may be added to a solution comprising the plurality of capture objects, or the plurality of capture objects may be added directly to or as a solution to the sample.

The terms “limit of detection” (or LOD) and “limit of quantification” (or LOQ) are given their ordinary meaning in the art. The LOD refers to the lowest analyte molecule concentration likely to be reliably distinguished from background noise and at which detection is feasible. The LOD as used herein is defined as three standard deviations (SD) above background noise. The LOQ refers to the lowest concentration at which the analyte molecule can not only be reliably detected but at which some predefined goals for bias and imprecision are met. Generally, as is used herein, the LOQ refers to the lowest concentration above the LOD wherein the coefficient of variation (CV) of the measured concentrations less than about 20%.

In some cases, an assay method employed has a limit of detection and/or a limit of quantification of less than about 500 pg/mL, 250 pg/mL, 100 pg/mL, 50 pg/mL, 40 pg/mL, 30 pg/mL, 20 pg/mL, 10 pg/mL 5 pg/mL, 4 pg/mL, 3 pg/mL, 2 pg/mL, 1 pg/mL, 0.8 pg/mL, 0.7 pg/mL, 0.6 pg/mL, 0.5 pg/mL, 0.4 pg/mL, 0.3 pg/mL, 0.2 pg/mL, 0.1 pg/mL, 0.05 pg/mL, 0.04 pg/mL, 0.02 pg/mL, 0.01 pg/mL, or less. In some cases, an assay method employed has a limit of quantification and/or a limit of detection between about 100 pg/mL and about 0.01 pg/mL, between about 50 pg/mL and about 0.02 pg/mL, between about 25 pg/mL and about 0.02 pg/mL, between about 10 pg/mL and about 0.02 pg/mL, between about 5 pg/mL and about 0.02 pg/mL, or between about 1 pg/mL and about 0.02 pg/mL. As will be understood by those of ordinary skill the art, the LOQ and/or LOD may differ for each assay method and/or each biomarker determined with the same assay.

In some embodiments, the concentration of analyte molecules (e.g., biomarker molecules) in the fluid sample that may be substantially accurately determined is less than about 5000 fM, less than about 3000 fM, less than about 2000 fM, less than about 1000 fM, less than about 500 fM, less than about 300 fM, less than about 200 fM, less than about 100 fM, less than about 50 fM, less than about 25 fM, less than about 10 fM, less than about 5 fM, less than about 2 fM, less than about 1 fM, less than about 0.5 fM, less than about 0.1 fM, or less. In some embodiments, the concentration of analyte molecules (e.g., biomarker molecules) in the fluid sample that may be substantially accurately determined is between about 5000 fM and about 0.1 fM, between about 3000 fM and about 0.1 fM, between about 1000 fM and about 0.1 fM, between about 1000 fM and about 1 fM, between about 100 fM and about 1 fM, between about 100 fM and about 0.1 fM, or the like. The concentration of analyte molecules or particles in a fluid sample may be considered to be substantially accurately determined if the measured concentration of the biomarker molecules in the fluid sample is within about 10% of the actual (e.g., true) concentration of the analyte molecule (e.g., biomarker molecules) in the fluid sample. In certain embodiments, the measured concentration of the analyte molecules (e.g., biomarker molecules) in the fluid sample may be within about 5%, within about 4%, within about 3%, within about 2%, within about 1%, within about 0.5%, within about 0.4%, within about 0.3%, within about 0.2% or within about 0.1%, of the actual concentration of the biomarker molecules in the fluid sample. In some cases, the measure of the concentration determined differs from the true (e.g., actual) concentration by no greater than about 20%, no greater than about 15%, no greater than 10%, no greater than 5%, no greater than 4%, no greater than 3%, no greater than 2%, no greater than 1%, or no greater than 0.5%. The accuracy of the assay method may be determined, in some embodiments, by determining the concentration of analyte molecules (e.g., biomarker molecules) in a fluid sample of a known concentration using the selected assay method.

In some embodiments, an assay method employs a step of spatially segregating analyte (e.g., biomarker) molecules into a plurality of locations to facilitate detection/quantification, such that each location comprises/contains either zero or one or more analyte (e.g., biomarker) molecules. Additionally, in some embodiments, the locations may be configured in a manner such that each location can be individually addressed. In some embodiments, a measure of the concentration of biomarker molecules in a fluid sample may be determined by detecting biomarker molecules immobilized with respect to a binding surface having affinity for at least one type of biomarker molecule. In certain embodiments the binding surface may form (e.g., a surface of a well/reaction vessel on a substrate) or be contained within (e.g., a surface of a capture object, such as a bead, contained within a well) one of a plurality of locations (e.g., a plurality of wells/reaction vessels) on a substrate (e.g., plate, dish, chip, optical fiber end, etc.). At least a portion of the locations may be addressed and a measure indicative of the number/percentage/fraction of the locations containing at least one analyte molecule (e.g., biomarker) molecule may be made. In some cases, based upon the number/percentage/fraction, a measure of the concentration of analyte molecule (e.g., average enzyme per bead) in the fluid sample may be determined. The measure of the concentration of analyte molecule (e.g., average enzyme per bead) in the fluid sample may be determined by a digital analysis method/system optionally employing Poisson distribution adjustment and/or based at least in part on a measured intensity of a signal, as will be known to those of ordinary skill in the art. In some cases, the assay methods and/or systems may be automated.

Certain methods and systems which employ spatially segregating analyte molecules (e.g., biomarkers) are known in the art, and are described in, for example, U.S. Pat. No. 8,460,879 (by Walt et al.); U.S. Pat. No. 8,460,878 (by Walt et al.); U.S. Pat. No. 8,492,098 (by Walt et al.); WO2009029073 (by Walt et al.); US20110195852 (by Walt et al.); US20100075862 (by Duffy et al.); US201000754072 (by Duffy et al.); US20100075439 (by Duffy et al.); WO2010/039179 (by Duffy et al.); US20100075355 (by Duffy et al.); US20110212848 (by Duffy et al.); WO 2011/109364 (by Duffy et al.); WO 2011/109372 (by Duffy et al.); US20110212462 (by Duffy et al.); WO 2011/109379 (by Rissin et al.); US20110212537 (by Duffy et al.); US20120196774 (by Fournier et al.); US 20110245097 (by Rissin et al.), U.S. Pat. No. 8,236,574 (by Duffy et al.); U.S. Pat. No. 8,415,171 (by Duffy et al.); WO2012/170776 (by Wilson et al.); WO2012/142300 (by Wilson et al.); WO2012/142301 (by Wilson et al.,); WO2014/113502 (by Duffy et al.); WO2014/183096 (by Rissin et al.); WO2016/115256 (by Duffy et al.); WO2016/130923 (by Pollock et al.), each of which are incorporated herein by reference in their entirety. These assay may also be referred to herein as digital ELISA assay, e.g., based on single molecule array (Simoa) technology. E.g., see also Rissin, et al., Nat Biotechnol, 28(6): p. 595-9 (2010); Rissin, et al., Anal Chem, 83(6): p. 2279-85 (2011); Kan, et al., Lab Chip, 2012. 12(5): p. 977-85.

Additional details of exemplary, non-limiting assay methods which comprise one or more steps of spatially segregating analyte (e.g., biomarker) molecules will now be described. In certain embodiments, a method for detection and/or quantifying analyte (e.g., biomarker) molecules in a sample comprises immobilizing a plurality of analyte (e.g., biomarker) molecules with respect to a plurality of capture objects (e.g., beads) that each include a binding surface having affinity for at least one type of analyte (e.g., biomarker). For example, the capture objects may comprise a plurality of beads comprising a plurality of capture components (e.g., an antibody having specific affinity for a biomarker of interest, etc.). At least some of the capture objects (e.g., at least some associated with at least one biomarker molecule) may be spatially separated/segregated into a plurality of locations, and at least some of the locations may be addressed/interrogated (e.g., using an imaging system). A measure of the concentration of analyte molecules in the fluid sample may be determined based on the information received when addressing the locations (e.g., using the information received from the imaging system and/or processed using a computer implemented control system). In some cases, a measure of the concentration may be based at least in part on the number of locations determined to contain a capture object that is or was associated with at least one analyte molecule. In other cases and/or under differing conditions, a measure of the concentration may be based at least in part on an intensity level of at least one signal indicative of the presence of a plurality of biomarker molecules and/or capture objects associated with an analyte molecule at one or more of the addressed locations.

In some embodiments, the number/percentage/fraction of locations containing a capture object but not containing a biomarker molecule may also be determined and/or the number/percentage/fraction of locations not containing any capture object may also be determined. In such embodiments, a measure of the concentration of analyte molecules in the fluid sample may be based at least in part on the ratio of the number of locations determined to contain a capture object associated with at least one analyte molecule to the total number of locations determined to contain a capture object not associated with an analyte molecule. In yet other embodiments, a measure of the concentration of analyte molecules in a fluid sample may be based at least in part on the ratio of the number of locations determined to contain a capture object and at least one analyte molecule to the total number of locations addressed and/or analyzed.

In certain embodiments, at least some of the plurality of capture objects (e.g., at least some associated with at least one analyte molecule) are spatially separated into a plurality of locations, for example, a plurality of reaction vessels in an array format. The plurality of reaction vessels may be formed in, on and/or of any suitable material, and in some cases, the reaction vessels can be sealed or may be formed upon the mating of a substrate with a sealing component, as discussed in more detail below. In certain embodiments, especially where quantization of the capture objects associated with at least one analyte molecule is desired, the partitioning of the capture objects can be performed such that at least some (e.g., a statistically significant fraction; e.g., as described in International Patent Publication No. WO2011/109364, filed Mar. 1, 2011, by Duffy et al.; U.S. Pat. No. 8,236,574, issued Aug. 7, 2012, by Duffy et al.; U.S. Pat. No. 8,415,171, issued Apr. 9, 2013, by Duffy et al.; International Patent Publication No. WO2011/109379, filed Mar. 1, 2011, by Rissin et al.) of the reaction vessels comprise at least one or, in certain cases, only one capture object associated with at least one analyte molecule and at least some (e.g., a statistically significant fraction) of the reaction vessels comprise an capture object not associated with any analyte molecules. The capture objects associated with at least one analyte molecule may be quantified in certain embodiments, thereby allowing for the detection and/or quantification of analyte molecules in the fluid sample by techniques described in more detail herein.

An exemplary assay method may proceed as follows. A sample fluid containing or suspected of containing analyte molecules is provided. An assay consumable comprising a plurality of assay sites is exposed to the sample fluid. In some cases, the analyte molecules are provided in a manner (e.g., at a concentration) such that a statistically significant fraction of the assay sites contain a single analyte molecule and a statistically significant fraction of the assay sites do not contain any analyte molecules. The assay sites may optionally be exposed to a variety of reagents (e.g., using a reagent loader) and or rinsed. The assay sites may then optionally be sealed and imaged (see, for example, U.S. patent application Ser. No. 13/035,472, filed Feb. 25, 2011, entitled “SYSTEMS, DEVICES, AND METHODS FOR ULTRA-SENSITIVE DETECTION OF MOLECULES OR PARTICLES,” by Fournier et al.). The images are then analyzed (e.g., using a computer implemented control system) such that a measure of the concentration of the analyte molecules in the fluid sample may be obtained, based at least in part, by determination of the number/fraction/percentage of assay sites which contain a analyte molecule and/or the number/fraction/percentage of sites which do not contain any analyte molecules. In some cases, the analyte molecules are provided in a manner (e.g., at a concentration) such that at least some assay sites comprise more than one analyte molecule. In such embodiments, a measure of the concentration of analyte molecules in the fluid sample may be obtained at least in part on an intensity level of at least one signal indicative of the presence of a plurality of analyte molecules at one or more of the assay sites

In some cases, the methods optionally comprise exposing the fluid sample to a plurality of capture objects, for example, beads. At least some of the analyte molecules are immobilized with respect to a bead. In some cases, the analyte molecules are provided in a manner (e.g., at a concentration) such that a statistically significant fraction of the beads associate with a single analyte molecule and a statistically significant fraction of the beads do not associate with any analyte molecules. At least some of the plurality of beads (e.g., those associated with a single analyte molecule or not associated with any analyte molecules) may then be spatially separated/segregated into a plurality of assay sites (e.g., of an assay consumable). The assay sites may optionally be exposed to a variety of reagents and/or rinsed. At least some of the assay sites may then be addressed to determine the number of assay sites containing a analyte molecule. In some cases, the number of assay sites containing a bead not associated with a analyte molecule, the number of assay sites not containing a bead and/or the total number of assay sites addressed may also be determined. Such determination(s) may then be used to determine a measure of the concentration of analyte molecules in the fluid sample. In some cases, more than one analyte molecule may associate with a bead and/or more than one bead may be present in an assay site. In some cases, the plurality analyte molecules may be exposed to at least one additional reaction component prior to, concurrent with, and/or following spatially separating at least some of the analyte molecules into a plurality of locations.

The analyte molecules may be directly detected or indirectly detected. In the case of direct detection, a analyte molecule may comprise a molecule or moiety that may be directly interrogated and/or detected (e.g., a fluorescent entity). In the case of indirect detection, an additional component is used for determining the presence of the analyte molecule. For example, the analyte molecules (e.g., optionally associated with a bead) may be exposed to at least one type of binding ligand. A “binding ligand,” is any molecule, particle, or the like which specifically binds to or otherwise specifically associates with a analyte molecule to aid in the detection of the analyte molecule. In certain embodiments, a binding ligand may be adapted to be directly detected (e.g., the binding ligand comprises a detectable molecule or moiety) or may be adapted to be indirectly detected (e.g., including a component that can convert a precursor labeling agent into a labeling agent). A component of a binding ligand may be adapted to be directly detected in embodiments where the component comprises a measurable property (e.g., a fluorescence emission, a color, etc.). A component of a binding ligand may facilitate indirect detection, for example, by converting a precursor labeling agent into a labeling agent (e.g., an agent that is detected in an assay). A “precursor labeling agent” is any molecule, particle, or the like, that can be converted to a labeling agent upon exposure to a suitable converting agent (e.g., an enzymatic component). A “labeling agent” is any molecule, particle, or the like, that facilitates detection, by acting as the detected entity, using a chosen detection technique. In some embodiments, the binding ligand may comprise an enzymatic component (e.g., horseradish peroxidase, beta-galactosidase, alkaline phosphatase, etc). A first type of binding ligand may or may not be used in conjunction with additional binding ligands (e.g., second type, etc.).

More than one type of binding may be employed in any given assay method, for example, a first type of binding ligand and a second type of binding ligand. In one example, the first type of binding ligand is able to associate with a first type of analyte molecule and the second type of binding ligand is able to associate with the first binding ligand. In another example, both a first type of binding ligand and a second type of binding ligand may associate with the same or different epitopes of a single analyte molecule, as described herein. In some embodiments, at least one binding ligand comprises an enzymatic component.

In some embodiments, a binding ligand and/or a analyte may comprise an enzymatic component. The enzymatic component may convert a precursor labeling agent (e.g., an enzymatic substrate) into a labeling agent (e.g., a detectable product). A measure of the concentration of analyte molecules in the fluid sample can then be determined based at least in part by determining the number of locations containing a labeling agent (e.g., by relating the number of locations containing a labeling agent to the number of locations containing a analyte molecule (or number of capture objects associated with at least one analyte molecule to total number of capture objects)). Non-limiting examples of enzymes or enzymatic components include horseradish peroxidase, beta-galactosidase, and alkaline phosphatase. Other non-limiting examples of systems or methods for detection include embodiments where nucleic acid precursors are replicated into multiple copies or converted to a nucleic acid that can be detected readily, such as the polymerase chain reaction (PCR), rolling circle amplification (RCA), ligation, Loop-Mediated Isothermal Amplification (LAMP), etc. Such systems and methods will be known to those of ordinary skill in the art, for example, as described in “DNA Amplification: Current Technologies and Applications,” Vadim Demidov et al., 2004.

Another exemplary embodiment of indirect detection is as follows. In some cases, the analyte molecules may be exposed to a precursor labeling agent (e.g., enzymatic substrate) and the enzymatic substrate may be converted to a detectable product (e.g., fluorescent molecule) upon exposure to a analyte molecule.

The assay methods and systems may employ a variety of different components, steps, and/or other aspects that will be known and understood by those of ordinary skill in the art. For example, a method may further comprise determining at least one background signal determination (e.g., and further comprising subtracting the background signal from other determinations), wash steps, and the like. In some cases, the assays or systems may include the use of at least one binding ligand, as described herein. In some cases, the measure of the concentration of analyte molecules in a fluid sample is based at least in part on comparison of a measured parameter to a calibration curve. In some instances, the calibration curve is formed at least in part by determination at least one calibration factor, as described above.

In certain embodiments, solubilized, or suspended precursor labeling agents may be employed, wherein the precursor labeling agents are converted to labeling agents which are insoluble in the liquid and/or which become immobilized within/near the location (e.g., within the reaction vessel in which the labeling agent is formed). Such precursor labeling agents and labeling agents and their use is described in commonly owned U.S. Patent Application Publication No. US-2010-0075862 (Ser. No. 12/236,484), filed Sep. 23, 2008, entitled “HIGH SENSITIVITY DETERMINATION OF THE CONCENTRATION OF ANALYTE MOLECULES OR PARTICLES IN A FLUID SAMPLE,” by Duffy et al., incorporated herein by reference.

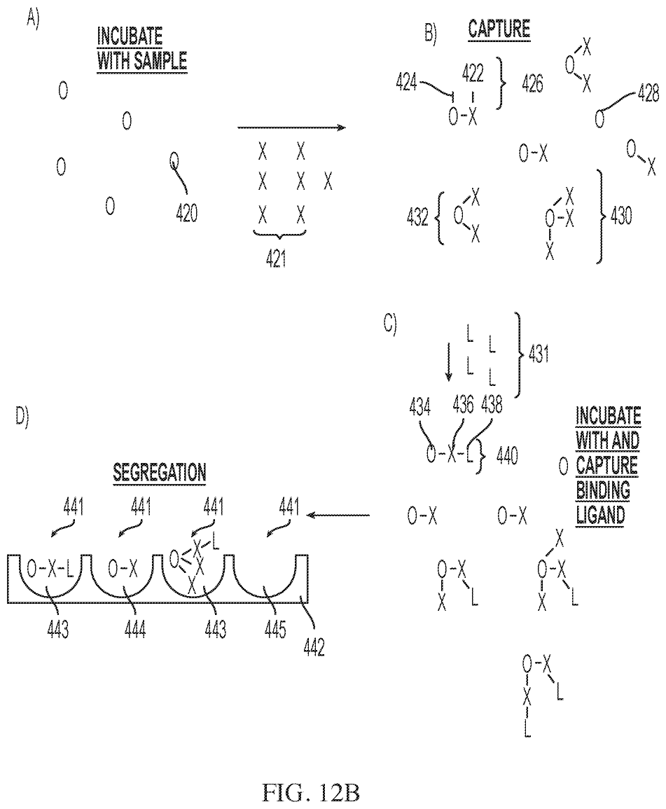

An exemplary embodiment of an assay method that may be used in certain embodiments of the invention is illustrated in FIG. 12A. A plurality of capture objects 402, are provided (step (A)). In this particular example, the plurality of capture objects comprises a plurality of beads. The beads are exposed to a fluid sample containing a plurality of analyte molecules 403 (e.g., beads 402 are incubated with analyte molecules 403). At least some of the analyte molecules are immobilized with respect to a bead. In this example, the analyte molecules are provided in a manner (e.g., at a concentration) such that a statistically significant fraction of the beads associate with a single analyte molecule and a statistically significant fraction of the beads do not associate with any analyte molecules. For example, as shown in step (B), analyte molecule 404 is immobilized with respect to bead 405, thereby forming complex 406, whereas some beads 407 are not associated with any analyte molecules. It should be understood, in some embodiments, more than one analyte molecule may associate with at least some of the beads, as described herein. At least some of the plurality of beads (e.g., those associated with a single analyte molecule or not associated with any analyte molecules) may then be spatially separated/segregated into a plurality of locations. As shown in step (C), the plurality of locations is illustrated as substrate 8 comprising a plurality of wells/reaction vessels 409. In this example, each reaction vessel comprises either zero or one beads. At least some of the reaction vessels may then be addressed (e.g., optically or via other detection means) to determine the number of locations containing a analyte molecule. For example, as shown in step (D), the plurality of reaction vessels are interrogated optically using light source 415, wherein each reaction vessel is exposed to electromagnetic radiation (represented by arrows 410) from light source 415. The light emitted (represented by arrows 411) from each reaction vessel is determined (and/or recorded) by detector 415 (in this example, housed in the same system as light source 415). The number of reaction vessels containing a analyte molecule (e.g., reaction vessels 412) is determined based on the light detected from the reaction vessels. In some cases, the number of reaction vessels containing a bead not associated with a analyte molecule (e.g., reaction vessel 413), the number of wells not containing a bead (e.g., reaction vessel 414) and/or the total number of wells addressed may also be determined. Such determination(s) may then be used to determine a measure of the concentration of analyte molecules in the fluid sample.

A non-limiting example of an embodiment where a capture object is associated with more than one analyte molecule is illustrated in FIG. 12B. A plurality of capture objects 420 are provided (step (A)). In this example, the plurality of capture objects comprises a plurality of beads. The plurality of beads is exposed to a fluid sample containing plurality of analyte molecules 421 (e.g., beads 420 are incubated with analyte molecules 421). At least some of the analyte molecules are immobilized with respect to a bead. For example, as shown in step (B), analyte molecule 422 is immobilized with respect to bead 424, thereby forming complex 426. Also illustrated is complex 430 comprising a bead immobilized with respect to three analyte molecules and complex 432 comprising a bead immobilized with respect to two analyte molecules. Additionally, in some cases, some of the beads may not associate with any analyte molecules (e.g., bead 428). The plurality of beads from step (B) is exposed to a plurality of binding ligands 431. As shown in step (C), a binding ligand associates with some of the analyte molecules immobilized with respect to a bead. For example, complex 440 comprises bead 434, analyte molecule 436, and binding ligand 438. The binding ligands are provided in a manner such that a statistically significant fraction of the beads comprising at least one analyte molecule become associated with at least one binding ligand (e.g., one, two, three, etc.) and a statistically significant fraction of the beads comprising at least one analyte molecule do not become associated with any binding ligands. At least a portion of the plurality of beads from step (C) are then spatially separated into a plurality of locations. As shown in step (D), in this example, the locations comprise a plurality of reaction vessels 441 on a substrate 442. The plurality of reaction vessels may be exposed to the plurality of beads from step (C) such at each reaction vessel contains zero or one beads. The substrate may then be analyzed to determine the number of reaction vessels containing a binding ligand (e.g., reaction vessels 443), wherein in the number may be related to a measure of the concentration of analyte molecules in the fluid sample. In some cases, the number of reaction vessels containing a bead and not containing a binding ligand (e.g., reaction vessel 444), the number of reaction vessels not containing a bead (e.g., reaction vessel 445), and/or the total number of reaction vessels addressed/analyzed may also be determined. Such determination(s) may then be used to determine a measure of the concentration of analyte molecules in the fluid sample.

In some embodiments, a plurality of locations may be addressed and/or a plurality of capture objects and/or species/molecules/particles of interest may be detected substantially simultaneously. “Substantially simultaneously” when used in this context, refers to addressing/detection of the locations/capture objects/species/molecules/particles of interest at approximately the same time such that the time periods during which at least two locations/capture objects/species/molecules/particles of interest are addressed/detected overlap, as opposed to being sequentially addressed/detected, where they would not. Simultaneous addressing/detection can be accomplished by using various techniques, including optical imaging techniques (e.g., CCD or CMOS detectors). Spatially segregating capture objects/species/molecules/particles into a plurality of discrete, resolvable locations, according to some embodiments facilitates substantially simultaneous detection by allowing multiple locations to be addressed substantially simultaneously. For example, for embodiments where individual species/molecules/particles are associated with capture objects that are spatially segregated with respect to the other capture objects into a plurality of discrete, separately resolvable locations during detection, substantially simultaneously addressing the plurality of discrete, separately resolvable locations permits individual capture objects, and thus individual species/molecules/particles (e.g., biomarker molecules) to be resolved. For example, in certain embodiments, individual molecules/particles of a plurality of molecules/particles are partitioned across a plurality of reaction vessels such that each reaction vessel contains zero or only one species/molecule/particle. In some cases, at least about 80%, at least about 85%, at least about 90%, at least about 95%, at least about 96%, at least about 97%, at least about 98%, at least about 99%, at least about 99.5% of all species/molecules/particles are spatially separated with respect to other species/molecules/particles during detection. A plurality of species/molecules/particles may be detected substantially simultaneously within a time period of less than about 1 second, less than about 500 milliseconds, less than about 100 milliseconds, less than about 50 milliseconds, less than about 10 milliseconds, less than about 1 millisecond, less than about 500 microseconds, less than about 100 microseconds, less than about 50 microseconds, less than about 10 microseconds, less than about 1 microsecond, less than about 0.5 microseconds, less than about 0.1 microseconds, or less than about 0.01 microseconds, less than about 0.001 microseconds, or less. In some embodiments, the plurality of species/molecules/particles may be detected substantially simultaneously within a time period of between about 100 microseconds and about 0.001 microseconds, between about 10 microseconds and about 0.01 microseconds, or less.

In some embodiments, the locations are optically interrogated. The locations exhibiting changes in their optical signature may be identified by a conventional optical train and optical detection system. Depending on the detected species (e.g., type of fluorescence entity, etc.) and the operative wavelengths, optical filters designed for a particular wavelength may be employed for optical interrogation of the locations. In embodiments where optical interrogation is used, the system may comprise more than one light source and/or a plurality of filters to adjust the wavelength and/or intensity of the light source. In some embodiments, the optical signal from a plurality of locations is captured using a CCD or CMOS camera.

In some embodiments of the present invention, the plurality of reaction vessels may be sealed (e.g., after the introduction of the analyte molecules, binding ligands, and/or precursor labeling agent), for example, through the mating of the second substrate and a sealing component. The sealing of the reaction vessels may be such that the contents of each reaction vessel cannot escape the reaction vessel during the remainder of the assay. In some cases, the reaction vessels may be sealed after the addition of the analyte molecules and, optionally, at least one type of precursor labeling agent to facilitate detection of the analyte molecules. For embodiments employing precursor labeling agents, by sealing the contents in some or each reaction vessel, a reaction to produce the detectable labeling agents can proceed within the sealed reaction vessels, thereby producing a detectable amount of labeling agents that is retained in the reaction vessel for detection purposes.

The plurality of locations may be formed may be formed using a variety of methods and/or materials. In some embodiments, the plurality of locations comprises a plurality of reaction vessels/wells on a substrate. In some cases, the plurality of reaction vessels is formed as an array of depressions on a first surface. In other cases, however, the plurality of reaction vessels may be formed by mating a sealing component comprising a plurality of depressions with a substrate that may either have a featureless surface or include depressions aligned with those on the sealing component. Any of the device components, for example, the substrate or sealing component, may be fabricated from a compliant material, e.g., an elastomeric polymer material, to aid in sealing. The surfaces may be or made to be hydrophobic or contain hydrophobic regions to minimize leakage of aqueous samples from the microwells. The reactions vessels, in certain embodiments, may be configured to receive and contain only a single capture object.

In some embodiments, the reaction vessels may all have approximately the same volume. In other embodiments, the reaction vessels may have differing volumes. The volume of each individual reaction vessel may be selected to be appropriate to facilitate any particular assay protocol. For example, in one set of embodiments where it is desirable to limit the number of capture objects used for analyte capture contained in each vessel to a small number, the volume of the reaction vessels may range from attoliters or smaller to nanoliters or larger depending upon the nature of the capture objects, the detection technique and equipment employed, the number and density of the wells on the substrate and the expected concentration of capture objects in the fluid applied to the substrate containing the wells. In one embodiment, the size of the reaction vessel may be selected such only a single capture object used for analyte capture can be fully contained within the reaction vessel (see, for example, U.S. Patent Application No. US 2011-0212848 (Ser. No. 12/731,130), filed Mar. 24, 2010, entitled “ULTRA-SENSITIVE DETECTION OF MOLECULES OR PARTICLES USING BEADS OR OTHER CAPTURE OBJECTS,” by Duffy et al.; International Patent Application Publication No. WO2011/109364 (International Patent Application No. PCT/US2011/026645), filed Mar. 1, 2011, entitled “ULTRA-SENSITIVE DETECTION OF MOLECULES OR PARTICLES USING BEADS OR OTHER CAPTURE OBJECTS,” by Duffy et al., each herein incorporated by reference).

In some embodiments, the reaction vessels may have a volume between about 1 femtoliter and about 1 picoliter, between about 1 femtoliters and about 100 femtoliters, between about 10 attoliters and about 100 picoliters, between about 1 picoliter and about 100 picoliters, between about 1 femtoliter and about 1 picoliter, or between about 30 femtoliters and about 60 femtoliters. In some cases, the reaction vessels have a volume of less than about 1 picoliter, less than about 500 femtoliters, less than about 100 femtoliters, less than about 50 femtoliters, or less than about 1 femtoliter. In some cases, the reaction vessels have a volume of about 10 femtoliters, about 20 femtoliters, about 30 femtoliters, about 40 femtoliters, about 50 femtoliters, about 60 femtoliters, about 70 femtoliters, about 80 femtoliters, about 90 femtoliters, or about 100 femtoliters.

The total number of locations and/or density of the locations employed in an assay (e.g., the number/density of reaction vessels in an array) can depend on the composition and end use of the array. For example, the number of reaction vessels employed may depend on the number of types of analyte molecule and/or binding ligand employed, the suspected concentration range of the assay, the method of detection, the size of the capture objects, the type of detection entity (e.g., free labeling agent in solution, precipitating labeling agent, etc.). Arrays containing from about 2 to many billions of reaction vessels (or total number of reaction vessels) can be made by utilizing a variety of techniques and materials. Increasing the number of reaction vessels in the array can be used to increase the dynamic range of an assay or to allow multiple samples or multiple types of analyte molecules to be assayed in parallel. The array may comprise between one thousand and one million reaction vessels per sample to be analyzed. In some cases, the array comprises greater than one million reaction vessels. In some embodiments, the array comprises between about 1,000 and about 50,000, between about 1,000 and about 1,000,000, between about 1,000 and about 10,000, between about 10,000 and about 100,000, between about 100,000 and about 1,000,000, between about 100,000 and about 500,000, between about 1,000 and about 100,000, between about 50,000 and about 100,000, between about 20,000 and about 80,000, between about 30,000 and about 70,000, between about 40,000 and about 60,000 reaction vessels. In some embodiments, the array comprises about 10,000, about 20,000, about 50,000, about 100,000, about 150,000, about 200,000, about 300,000, about 500,000, about 1,000,000, or more, reaction vessels.

The array of reaction vessels may be arranged on a substantially planar surface or in a non-planar three-dimensional arrangement. The reaction vessels may be arrayed in a regular pattern or may be randomly distributed. In a specific embodiment, the array is a regular pattern of sites on a substantially planar surface permitting the sites to be addressed in the X-Y coordinate plane.

In some embodiments, the reaction vessels are formed in a solid material. As will be appreciated by those in the art, the number of potentially suitable materials in which the reaction vessels can be formed is very large, and includes, but is not limited to, glass (including modified and/or functionalized glass), plastics (including acrylics, polystyrene and copolymers of styrene and other materials, polypropylene, polyethylene, polybutylene, polyurethanes, cyclic olefin copolymer (COC), cyclic olefin polymer (COP), Teflon®, polysaccharides, nylon or nitrocellulose, etc.), elastomers (such as poly(dimethyl siloxane) and poly urethanes), composite materials, ceramics, silica or silica-based materials (including silicon and modified silicon), carbon, metals, optical fiber bundles, or the like. In general, the substrate material may be selected to allow for optical detection without appreciable autofluorescence. In certain embodiments, the reaction vessels may be formed in a flexible material.

A reaction vessel in a surface (e.g., substrate or sealing component) may be formed using a variety of techniques known in the art, including, but not limited to, photolithography, stamping techniques, molding techniques, etching techniques, or the like. As will be appreciated by those of the ordinary skill in the art, the technique used can depend on the composition and shape of the supporting material and the size and number of reaction vessels.

In a particular embodiment, an array of reaction vessels is formed by creating microwells on one end of a fiber optic bundle and utilizing a planar compliant surface as a sealing component. In certain such embodiments, an array of reaction vessels in the end of a fiber optic bundle may be formed as follows. First, an array of microwells is etched into the end of a polished fiber optic bundle. Techniques and materials for forming and etching a fiber optic bundle are known to those of ordinary skill in the art. For example, the diameter of the optical fibers, the presence, size and composition of core and cladding regions of the fiber, and the depth and specificity of the etch may be varied by the etching technique chosen so that microwells of the desired volume may be formed. In certain embodiments, the etching process creates microwells by preferentially etching the core material of the individual glass fibers in the bundle such that each well is approximately aligned with a single fiber and isolated from adjacent wells by the cladding material. Potential advantages of the fiber optic array format is that it can produce thousands to millions of reaction vessels without complicated microfabrication procedures and that it can provide the ability to observe and optically address many reaction vessels simultaneously.

Each microwell may be aligned with an optical fiber in the bundle so that the fiber optic bundle can carry both excitation and emission light to and from the wells, enabling remote interrogation of the well contents. Further, an array of optical fibers may provide the capability for simultaneous or non-simultaneous excitation of molecules in adjacent vessels, without signal “cross-talk” between fibers. That is, excitation light transmitted in one fiber does not escape to a neighboring fiber.

Alternatively, the equivalent structures of a plurality of reaction vessels may be fabricated using other methods and materials that do not utilize the ends of an optical fiber bundle as a substrate. For example, the array may be a spotted, printed or photolithographically fabricated substrate produced by techniques known in the art; see for example WO95/25116; WO95/35505; PCT US98/09163; U.S. Pat. Nos. 5,700,637, 5,807,522, 5,445,934, 6,406,845, and 6,482,593. In some cases, the array may be produced using molding, embossing, and/or etching techniques as will be known to those of ordinary skill in the art.

In some embodiments, the plurality of locations may not comprise a plurality of reaction vessels/wells. For example, in embodiments where capture objects are employed, a patterned substantially planar surface may be employed and the patterned areas form a plurality of locations. In some cases, the patterned areas may comprise substantially hydrophilic surfaces which are substantially surrounded by substantially hydrophobic surfaces. In certain embodiments, a plurality of capture objects (e.g., beads) may be substantially surrounded by a substantially hydrophilic medium (e.g., comprising water), and the beads may be exposed to the patterned surface such that the beads associate in the patterned areas (e.g., the hydrophilic locations on the surface), thereby spatially segregating the plurality of beads. For example, in one such embodiment, a substrate may be or include a gel or other material able to provide a sufficient barrier to mass transport (e.g., convective and/or diffusional barrier) to prevent capture objects used for analyte capture and/or precursor labeling agent and/or labeling agent from moving from one location on or in the material to another location so as to cause interference or cross-talk between spatial locations containing different capture objects during the time frame required to address the locations and complete the assay. For example, in one embodiment, a plurality of capture objects is spatially separated by dispersing the capture objects on and/or in a hydrogel material. In some cases, a precursor labeling agent may be already present in the hydrogel, thereby facilitating development of a local concentration of the labeling agent (e.g., upon exposure to a binding ligand or analyte molecule carrying an enzymatic component). As still yet another embodiment, the capture objects may be confined in one or more capillaries. In some cases, the plurality of capture objects may be absorbed or localized on a porous or fibrous substrate, for example, filter paper. In some embodiments, the capture objects may be spatially segregated on a uniform surface (e.g., a planar surface), and the capture objects may be detected using precursor labeling agents which are converted to substantially insoluble or precipitating labeling agents that remain localized at or near the location of where the corresponding capture object is localized. The use of such substantially insoluble or precipitating labeling agents is described herein. In some cases, single analyte molecules may be spatially segregated into a plurality of droplets. That is, single analyte molecules may be substantially contained in a droplet containing a first fluid. The droplet may be substantially surrounded by a second fluid, wherein the second fluid is substantially immiscible with the first fluid.

In some embodiments, during the assay, at least one washing step may be carried out. In certain embodiments, the wash solution is selected so that it does not cause appreciable change to the configuration of the capture objects and/or analyte molecules and/or does not disrupt any specific binding interaction between at least two components of the assay (e.g., a capture component and a analyte molecule). In other cases, the wash solution may be a solution that is selected to chemically interact with one or more assay components. As will be understood by those of ordinary skill in the art, a wash step may be performed at any appropriate time point during the inventive methods. For example, a plurality of capture objects may be washed after exposing the capture objects to one or more solutions comprising analyte molecules, binding ligands, precursor labeling agents, or the like. As another example, following immobilization of the analyte molecules with respect to a plurality of capture objects, the plurality of capture objects may be subjected to a washing step thereby removing any analyte molecules not specifically immobilized with respect to a capture object.

Other assay methods in addition to those described herein are known in the art and may be used in connection with the inventive methods. For example, various analyzers are commercially available for the determination of the concentration of analyte. The assay methods employed should meet the algorithm requirements for LOD and LOQ.

The following examples are intended to illustrate certain embodiments of the present invention, but do not exemplify the full scope of the invention.

### EXAMPLES

### Example 1

This example describes the effect of 16.7 wt. % of sucrose on signal loss due to storage of an assay mixture. Assay mixtures that were exposed to 16.7 wt. % of sucrose prior to and after desolvation had less signal loss than assay mixtures that were not exposed to sucrose.

A PSA detection assay was used to investigate the effect on sucrose on signal loss. Briefly, paramagnetic beads conjugated with anti-PSA capture antibodies were exposed to solutions containing 30 pg/mL PSA. The captured PSA molecules were then labeled with anti-PSA detection antibody (“DetAb”) in the presence or absence of 16.7 wt. % of sucrose. The beads were pelleted and the solution aspirated and the beads were stored in pellet form for various times. Then, the pellets were exposed to a solution containing enzyme conjugate (“SbG”) and 16.7 wt. % sucrose. Bead pellets were resuspended in enzyme substrate and each were analyzed using single molecule arrays (Simoa). The average fluorescence intensity of beads (I_{bead}) in each pellet was then determined as described in Rissin et al., Anal. Chem., 2011 83(6), 2279-2285.

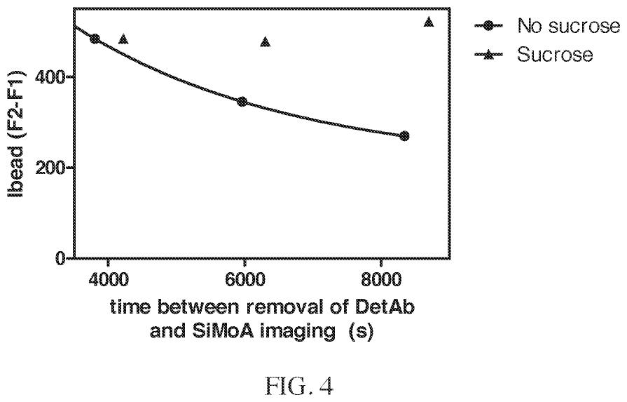

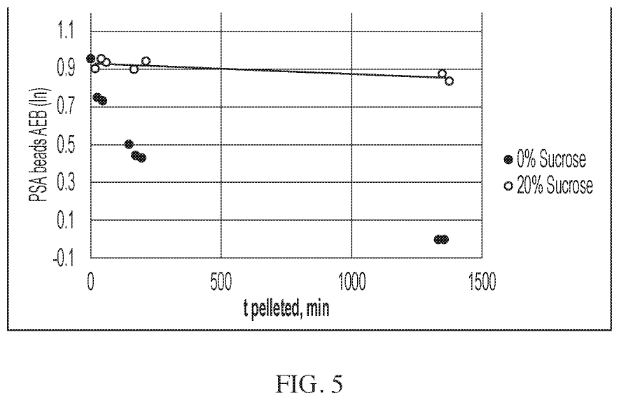

FIG. 4 shows a graph of the I_{bead} signal derived from individual pellets as a function of the storage time for the assay mixtures in the presence or absence of sucrose. As shown in FIG. 4, the DetAb assay mixture including sucrose did not exhibit a loss of signal. The assay mixture not including sucrose did exhibit a loss of signal, and an exponential fit to these data (solid line) showed a rate equal to the previously determined dissociation rate of this detection antibody. These data indicate that the 16.7 wt. % of sucrose reduces the drop of signal due to the dissociation of the detection antibody from the captured PSA molecules.

### Example 2

This example describes the effect of 20 wt. % of sucrose on signal loss due to storage of an assay mixture. Assay mixtures that were exposed to 20 wt. % of sucrose prior to storage had less signal loss as a function of time than assay mixtures that were not exposed to sucrose.

A prostate specific antigen (PSA) detection assay was used to investigate the effect of sucrose on signal loss as a function of time. Briefly, paramagnetic beads conjugated with anti-PSA capture antibodies were exposed to solutions containing approximately 10 pg/mL PSA. The captured PSA molecules were then labeled with anti-PSA detection antibody (“DetAb”). Then, the pellets were exposed to a solution containing enzyme conjugate (“SbG”) and washed in buffer. After the final wash, the beads were resuspended in a buffer that contained 20% sucrose, pelleted on a magnet, and the supernatant buffer was removed and the pellets dried. The bead pellets were stored for various times and then re-suspended in enzyme substrate. The signal was measured immediately after resuspension in enzyme substrate using single molecule arrays (Simoa). The average number of enzymes per bead (AEB) of each pellet was then determined as described in Rissin et al., Anal. Chem., 2011 83(6), 2279-2285. The AEB signal was measured for the different drying times over about a 24 hour time period as shown in Table 1.

FIG. 5 shows a graph of the signal versus the storage time for assay mixtures that were desolvated and stored with sucrose and assay mixtures that were desolvated and stored without sucrose. As shown in Table 1 and FIG. 5, the assay mixture including sucrose showed significantly reduced signal loss during storage.

TABLE 1
Signal as a function of storage time (t)
for 0 wt. % and 20 wt. % of sucrose

| | |
|---|---|
| 0% Sucrose | 20% Sucrose |

| | % AEB | | % AEB |
|---|---|---|---|
| t, min | Maintained | t, min | Maintained |
| 0 | 100% | 17 | 94% |
| 24 | 79% | 41 | 100% |
| 47 | 77% | 61 | 98% |
| 149 | 53% | 168 | 94% |
| 174 | 46% | 191 | — |
| 196 | 45% | 214 | 98% |
| 1337 | 0% | 1354 | 91% |
| 1359 | 0% | 1377 | 87% |

### Example 3

This example describes the effect of 16.7 wt. % of sucrose on enzymatic activity after storage of an assay mixture. Assay mixtures that were exposed to 16.7 wt. % of sucrose prior to and after desolvation had little or no reduction in enzymatic activity, whereas assay mixtures that were not exposed to sucrose had a significant reduction in enzymatic activity during storage.

An enzyme detection assay was used to investigation of the effect on sucrose on signal loss. Briefly, paramagnetic beads presenting biotin groups were incubated with solutions containing streptavidin-beta-galactosidase (“SbG”), washed, and then pelleted in the presence or absence of 16.7 wt. % of sucrose, and stored for various periods of time. After storage, the pellets were re-suspended in enzyme substrate and the enzymatic activity was immediately measured by single molecule arrays (Simoa). The average fluorescence intensity of beads (I_{bead}) in each pellet was determined as described in Rissin et al., Anal. Chem., 2011 83(6), 2279-2285.

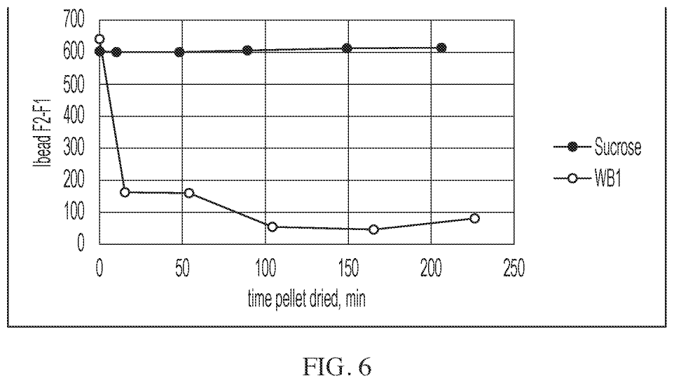

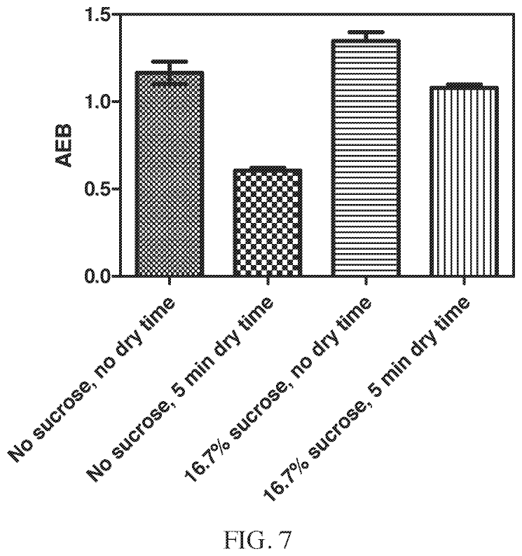

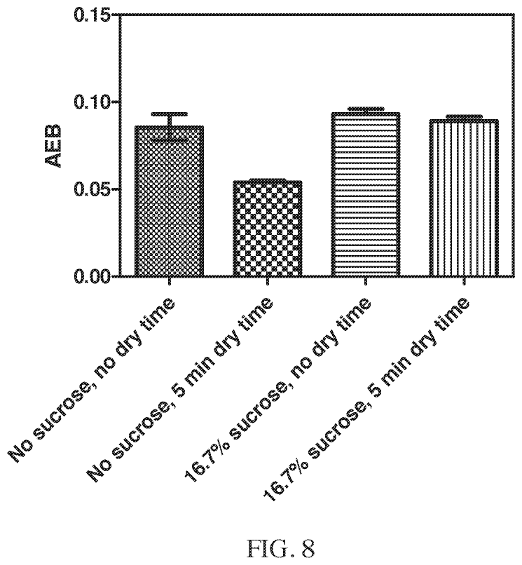

FIG. 6 shows a graph of the average intensity for the active beads in each pellet versus the storage time for the pelleted assay mixtures with and without sucrose. As shown in FIG. 6, the enzymatic activity of SbG when stored in the presence of sucrose was relatively constant over several hours, whereas the enzymatic activity of SbG when stored in the absence of sucrose decreased dramatically after less than about 30 minutes. Since the dissociation rate of biotin and streptavidin is very low, the difference in signal between sucrose and non-sucrose sample was likely due to differences in enzymatic activity (i.e., sucrose reduced denaturation of the enzyme in the dried state) and not dissociation.

### Example 4

This example describes the use of wash buffer containing 16.7 wt. % of sucrose on signal loss when the assay mixture is left dry between wash steps. Sucrose in the wash buffer reduced signal loss due to dissociation and denaturation when the assay mixture was left dry between wash steps. Assays that did not use sucrose in the wash buffer had a significant signal loss.

A PSA detection assay was used to investigate the effect on sucrose on signal loss. Briefly, paramagnetic beads conjugated with anti-PSA capture antibodies were exposed to solutions containing 15 pg/mL (as shown in FIG. 7) or 0.7 pg/mL (as shown in FIG. 8) PSA. The captured PSA molecules were then labeled sequentially with anti-PSA detection antibody (“DetAb”) and enzyme conjugate (“SbG”) both in the presence of 16.7 wt. % of sucrose. After each incubation with PSA solution, DetAb, or SbG, the beads were washed under four different conditions: 1) DetAb and SbG reagents added immediately after removal of wash buffer, i.e., no drying time, with no sucrose in the wash buffer; 2) DetAb and SbG reagents added 5 min after removal of wash buffer, i.e., 5 min drying time, with no sucrose in the wash buffer; 3) DetAb and SbG reagents added immediately after removal of wash buffer, i.e., no drying time, with 16.7% wt. sucrose in the wash buffer; 4) DetAb and SbG reagents added 5 min after removal of wash buffer, i.e., 5 min drying time, with 16.7% wt. sucrose in the wash buffer. The beads were resuspended in enzyme substrate at the end of the experiment and the signal was measured using single molecule arrays (Simoa). The average number of enzymes per bead (AEB) of each pellet was then determined as described in Rissin et al., Anal. Chem., 2011 83(6), 2279-2285.

FIG. 7 and FIG. 8 show bar charts of AEB for the four drying conditions for a sample containing relatively high analyte concentration (15 pg/mL) and a relatively low analyte concentration (0.7 pg/mL), respectively. The data are averages of 4 replicates of the same condition, and the error bars represent one standard deviation of the mean. Regardless of the analyte concentration, the signal loss when the beads were dried for 5 min was lower for assays that used sucrose in the wash buffer. The signal dropped by 37% and 40% compared to the control for low analyte concentration and high analyte concentrations, respectively, when sucrose was not included in the wash buffer.

### Example 5

This example describes the effect of the weight percentage of sucrose on the signal produced without a drying step, e.g., the minimum time required to process bead pellets. The signal increased with the weight percentage of sucrose used during the final pelleting step before the signal was determined. The signal produced using 30 wt. % of sucrose was about 5 times the signal produced when no sucrose was used.

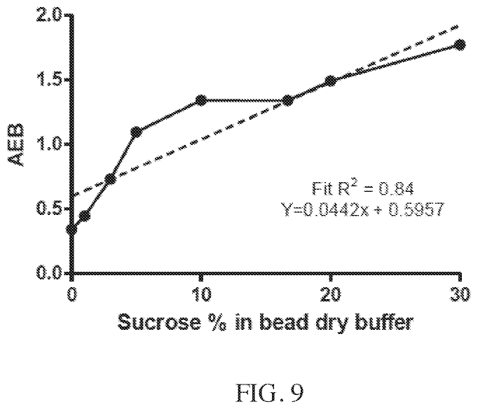

A PSA detection assay was used to investigate the effect of the weight percentage of sucrose on the signal produced after the assay beads were pelleted before the signal was measured. Briefly, paramagnetic beads conjugated with anti-PSA capture antibodies were exposed to solutions containing approximately 30 pg/mL PSA. The captured PSA molecules were then labeled with anti-PSA detection antibody (“DetAb”). Then, the pellets were exposed to a solution containing enzyme conjugate (“SbG”) and washed in buffer. After the final wash, the beads were resuspended in a buffer that contained varying concentrations (0-30%) of sucrose, pelleted on a magnet, and the supernatant buffer was removed. The bead pellets were immediately re-suspended in enzyme substrate. The signal was measured immediately after resuspension in enzyme substrate using single molecule arrays (Simoa). The average number of enzymes per bead (AEB) of each pellet was then determined as described in Rissin et al., Anal. Chem., 2011 83(6), 2279-2285. FIG. 9 shows a graph of the AEB versus the weight percentage of sucrose. As shown in FIG. 9, the signal increased with increasing sucrose concentration even with the minimum drying time possible.

### Example 6

This example describes the effect of the weight percentage of sucrose on aggregation during suspension of dried beads. Sucrose solution containing at least 10 wt. % significantly reduced aggregation of the beads.

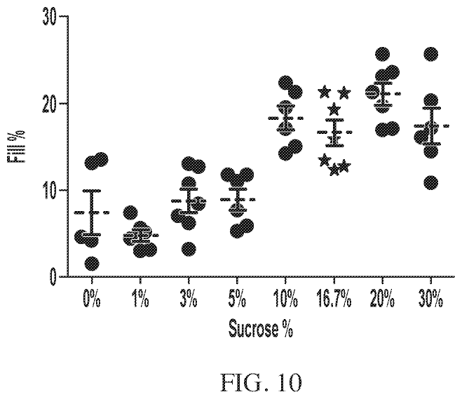

A PSA detection assay was used to investigate the effect of the weight percentage of sucrose on aggregation of the beads. Briefly, paramagnetic beads conjugated with anti-PSA capture antibodies were exposed to solutions containing approximately 30 pg/mL PSA. The captured PSA molecules were then labeled with anti-PSA detection antibody (“DetAb”). Then, the pellets were exposed to a solution containing enzyme conjugate (“SbG”) and washed in buffer. After the final wash, the beads were resuspended in a buffer that contained varying concentrations (0-30%) of sucrose, pelleted on a magnet, and the supernatant buffer was removed. The bead pellets were then dried in air for times varying from 0 to 200 min, re-suspended in enzyme substrate, and the signal was measured using single molecule arrays (Simoa). The bead fill of arrays (i.e., % Fill, equal to the fraction of the wells in the array containing a bead) from each pellet was then determined as described in Rissin et al., Anal. Chem., 2011 83(6), 2279-2285. FIG. 10 shows a graph of bead fill percentage versus the weight percentage of sucrose used in the re-suspension step for all drying times. Bead fill is reduced if beads become aggregated during drying as fewer single beads will be available to fit into the wells. As shown in FIG. 10, bead fill was significantly lower at 5% sucrose and below, suggesting that aggregation was significantly decreased when at least 10 wt. % of sucrose was used to suspend the beads.

### Example 7

This example describes the effect of various weight percentages of sucrose on signal loss as a function of storage time. The use of sucrose in the assay at all tested weight percentages significantly reduced signal loss over time compared to the absence of sucrose. Sucrose solutions containing between about 10 wt. % and about 30 wt. % had the least signal loss.

A PSA detection assay was used to investigation of the effect of the weight percentage of sucrose on signal loss as a function of time. Briefly, paramagnetic beads conjugated with anti-PSA capture antibodies were exposed to solutions containing approximately 30 pg/mL PSA. The captured PSA molecules were then labeled with anti-PSA detection antibody (“DetAb”). Then, the pellets were exposed to a solution containing enzyme conjugate (“SbG”) and washed in buffer. After the final wash, the beads were resuspended in a buffer that contained varying concentrations (0-30%) of sucrose, pelleted on a magnet, and the supernatant buffer was removed. The bead pellets were then dried in air for times varying from 0 to 200 min, re-suspended in enzyme substrate, and the signal was measured using single molecule arrays (Simoa). The average number of enzymes per bead (AEB) of each pellet was then determined as described in Rissin et al., Anal. Chem., 2011 83(6), 2279-2285. The normalized signal after 200 minutes of storage for various weight percentages of sucrose is shown in Table 2.

TABLE 2
Normalized signal after 200 minutes for
various weight percentages of sucrose

| | @t = 200 min |
|---|---|
| | Normalized |
| % Sucrose | AEB loss |
| 0 | 0.238 |
| 1 | 0.749 |
| 3 | 0.713 |
| 5 | 0.765 |
| 10 | 0.804 |
| 16.7 | 1.040 |
| 20 | 0.932 |
| 30 | 0.860 |

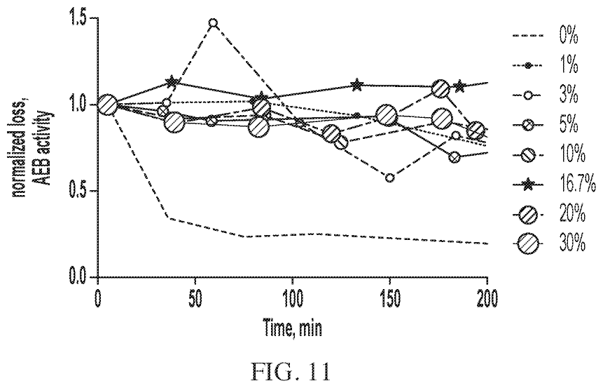

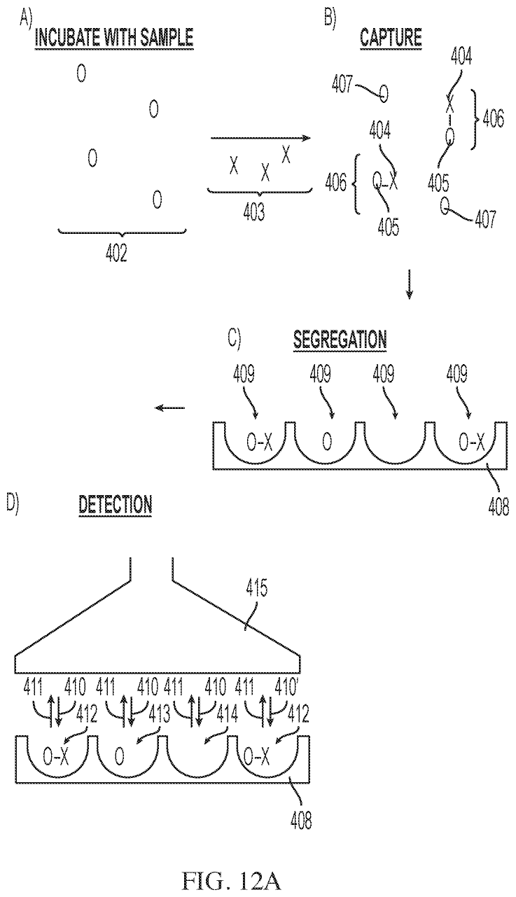

FIG. 11 shows a graph of normalized AEB versus the storage time for various weight percentages of sucrose. As shown in FIG. 11, signal loss was reduced at all weight percentages of sucrose compared to no sucrose. Sucrose solutions containing between about 10 wt. % and about 30 wt. % had the least signal loss.

### Example 8

This example describes the effect of various weight percentages of sucrose and various weight percentages of trehalose on signal loss after 200 minutes of storage. Trehalose and sucrose both significantly reduced signal loss compared to the absence of sucrose. Trehalose and sucrose produced a similar reduction in signal loss.

A PSA detection assay was used to investigate the effect of the weight percentage of trehalose and sucrose on signal loss after 200 minutes of storage. Briefly, paramagnetic beads conjugated with anti-PSA capture antibodies were exposed to solutions containing approximately 30 pg/mL PSA. The captured PSA molecules were then labeled with anti-PSA detection antibody (“DetAb”). Then, the pellets were exposed to a solution containing enzyme conjugate (“SbG”) and washed in buffer. After the final wash, the beads were resuspended in a buffer that contained varying concentrations of either sucrose or trehalose, pelleted on a magnet, and the supernatant buffer was removed. The bead pellets were then dried in air and stored for 200 min, re-suspended in enzyme substrate, and the signal was measured using single molecule arrays (Simoa). The average number of enzymes per bead (AEB) of each pellet was then determined as described in Rissin et al., Anal. Chem., 2011 83(6), 2279-2285. The normalized AEB signal for various sucrose and trehalose concentrations after 200 minutes of storage compared to 0 min of storage time is shown in Table 3.

TABLE 3
Signal after 200 minutes for various weight
percentages of sucrose and trehalose

| | | Ratio of AEB @ |
|---|---|---|
| | % (Wsugar/ | 200 min compared |
| Sugar | Wtotal) | to 0 min |
| None | 0 | 0.32 |
| Sucrose | 1 | 0.74 |
| Sucrose | 3 | 0.68 |
| Sucrose | 5 | 0.78 |
| Sucrose | 10 | 0.88 |
| Sucrose | 16.7 (Mar. 31, 2017) | 1.06 |
| Sucrose | 20 | 0.86 |
| Sucrose | 30 | 0.96 |
| Trehalose | 5 | 0.80 |
| Trehalose | 10 | 0.80 |
| Trehalose | 15 | 1.16 |
| Trehalose | 16.7 | 0.84 |
| Trehalose | 20 | 0.98 |
| Sucrose | 16.7 (Apr. 14, 2017) | 0.94 |

As shown in Table 3, sucrose and trehalose reduced signal loss at all concentrations. Trehalose and sucrose had a similar reduction in signal loss.

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present invention.

The indefinite articles “a” and “an,” as used herein in the specification and in the claims, unless clearly indicated to the contrary, should be understood to mean “at least one.”

In the claims, as well as in the specification above, all transitional phrases such as “comprising,” “including,” “carrying,” “having,” “containing,” “involving,” “holding,” and the like are to be understood to be open-ended, i.e., to mean including but not limited to. Only the transitional phrases “consisting of” and “consisting essentially of” shall be closed or semi-closed transitional phrases, respectively.

What is claimed is:

1. A method for perform an assay, comprising:
combining a stabilizing agent and an assay composition to form a first liquid mixture comprising a liquid, wherein the assay composition comprises detection molecules associated with analyte molecules;
separating at least a portion of a liquid from at least a portion of the first liquid mixture to form a pellet and a separated liquid, wherein the pellet comprises at least a portion of the assay composition and at least a portion of the stabilizing agent; and
removing greater than or equal to about 95% of the separated liquid from the pellet, wherein the stabilizing agent comprises a molecule having greater than four hydroxyl groups.

2. A method for preventing or reducing signal decay in an assay, comprising:
combining a stabilizing agent with an assay composition to form a first liquid mixture, wherein the assay composition comprises detection molecules associated with analyte molecules;
removing greater than or equal to about 95% of a liquid from the first liquid mixture to form a concentrated composition comprising the assay composition and the stabilizing agent;
storing the concentrated composition for at least 1 minute;
reconstituting the concentrated composition to form a second liquid mixture; and
measuring a signal used to determine a concentration of the analyte molecules in the second liquid mixture, wherein a magnitude of the signal measured in the measuring step is greater than or equal to about 10% of the magnitude of a signal used to determine a concentration of the analyte molecules of the analyte molecules in an otherwise identically prepared second liquid mixture except not subjected to the storage step.

3. A method of any preceding claim, wherein the assay composition further comprises a plurality of capture objects associated with a plurality of analyte molecules.

4. A method of any preceding claim, wherein the pellet or concentrated composition comprises at least 1000 capture objects, or at least 2000 capture objects, or at least 3000 capture objects, or at least 5000 capture objects, or at least 10,000 capture objects.

5. A method of claim 3 or 4, wherein the plurality of capture objects comprise a plurality of beads.

6. The method of any preceding claim, wherein the stabilizing agent is a disaccharide.

7. The method of any preceding claim, wherein the stabilizing agent has a molecular weight of less than or equal to about 500 g/mol.

8. The method of any preceding claim, wherein the stabilizing agent is a sucrose, trehalose, or combinations thereof.

9. The method of any preceding claim, wherein the stabilizing agent comprises a molecule having greater than six hydroxyl groups.

10. The method of any preceding claim, wherein the stabilizing agent alters a dissociation rate between the detection molecules and the analyte molecules in the pellet.

11. The method of any preceding claim, wherein a weight percentage of the stabilizing agent in the first liquid mixture is greater than or equal to about 1 wt. % and less than or equal to about 30 wt %.

12. The method of any preceding claim, wherein a weight percentage of the stabilizing agent in the first liquid mixture is greater than or equal to about 10 wt. % and less than or equal to about 30 wt %.

13. The method of any preceding claim, wherein the concentration of analyte molecules in the assay composition is less than about 50×10⁻¹⁵ M.

14. The method of any preceding claim, wherein the removing step comprises removing greater than or equal to about 98% of the separated liquid from the pellet.

15. The method of any preceding claim, wherein the removing step comprises aspirating greater than or equal to about 95% of the separated liquid from the pellet.

16. The method of any one of claims 1 and 3-15, wherein the removing step comprises exposing the pellet to reduced pressure.

17. The method of any one of claims 1 and 3-16, wherein the removing step comprises exposing the pellet to reduced pressure in the presence of a desiccant.

18. The method of claim 16 or 17, wherein the reduced pressure is less than or equal to about a vapor pressure of the separated liquid.

19. The method of any one of claims 1 and 3-18, wherein the separating step comprises separating the liquid from the first liquid mixture to form the pellet.

20. The method of any one of claims 1 and 3-19, comprising storing the pellet for at least 5 minutes.

21. The method of any preceding one of claims 1 and 3-20, comprising storing the pellet for at least 12 hours.

22. The method of any preceding claim, wherein less than about 5% of the analyte molecules in the composition have dissociated from the detection molecules after the storing step.

23. The method of any one of claims 1 and 3-22, comprising suspending the pellet in a second liquid mixture comprising the stabilizing agent to form a third liquid mixture.

24. The method of any one of claims 2-15 and 22, wherein a weight percentage of the stabilizing agent in the second liquid mixture is greater than or equal to about 1 wt. % and less than or equal to about 30 wt %.

25. The method of any one of claims 2-15 and 22-24, wherein a weight percentage of the stabilizing agent in the second liquid mixture is greater than or equal to about 10 wt. % and less than or equal to about 30 wt %.

26. The method of any one of claims 23-25, comprising measuring a signal indicative of a concentration of the analyte molecules in the third liquid mixture.

27. The method of any preceding claim, wherein the detection molecules comprises a protein.

28. The method of claim 27, wherein the protein is an enzyme.

29. The method of any preceding claim, wherein the analyte molecule is a biomarker.

30. A method of any one of claims 2-15, 22, and 24-29, wherein the concentrated composition is stored for at least 5 minutes, at least 10 minutes, at least 15 minutes, at least 30 minutes, at least 60 minutes, at least 2 hours, at least 4 hours.

31. The method any one of claims 2-15, 22, and 24-30, wherein the signal used to determine the concentration of the analyte molecules in the second liquid mixture is determined using a digital ELISA assay.
